(19) United States
(12) Patent Application Publication
(10) Pub. No.: US 2023/0096620 A1
(43) Pub. Date: Mar. 30, 2023

(54) ALPHA4BETA7 INHIBITOR AND IL-23 INHIBITOR COMBINATION THERAPY

(71) Applicant: Millennium Pharmaceuticals, Inc., Cambridge, MA (US)

(72) Inventors: Jorge Rodrigo MORA, Newton, MA (US); Michele Luise Gerber, Cambridge, MA (US); Neil Bradford Lineberry, Arlington, MA (US); Heath Michael Guay, Hopkinton, MA (US)

(21) Appl. No.: 17/604,324

(22) PCT Filed: Apr. 17, 2020
(86) PCT No.: PCT/US20/28861

§ 371 Date: Oct. 15, 2021

Related U.S. Application Data
(60) Provisional application No. 62/835,349, filed on Apr. 17, 2019.

(51) Int. Cl. C07K 16/28; C07K 16/24; A61P 1/12; A61P 37/06

(52) U.S. Cl. CPC C07K 16/2839; C07K 16/244; A61P 1/12; A61P 37/06; C07K 2317/24

(57) ABSTRACT

Provided herein are combination therapies comprising an alpha4beta7 inhibitor, e.g., an anti-alpha4beta7 antibody, e.g., vedolizumab, and an IL-23 inhibitor, e.g., an anti-IL-23 antibody.

### RELATED APPLICATIONS

This application is a U.S. National Phase application under 35 U.S.C. § 371 of International Appln. No. PCT/US2020/028861, filed on Apr. 17, 2020, which claims priority to U.S. Provisional Patent Appln. No. 62/835,349, filed on Apr. 17, 2019, the entire contents of which are incorporated by reference herein.

### SEQUENCE LISTING

This application contains a Sequence Listing which is submitted herewith in electronically readable format. The Sequence Listing file was created on Apr. 14, 2020, is named “2021_10_14_SL_T103022_1100 US.TXT” and its size is 24 kb. The entire contents of the Sequence Listing in the sequencelisting.txt file are incorporated by reference herein.

### FIELD OF THE INVENTION

The present invention relates to methods and compositions relating to a combination therapy comprising an α4β7 inhibitor, e.g., an anti-α4β7 antibody, (e.g., vedolizumab), and an IL-23 inhibitor, e.g., an anti-IL-23 antibody.

### BACKGROUND

Interleukin-23 (IL-23) is a heterodimeric cytokine composed of a p40 subunit and a p19 subunit. IL-23 is generally produced by antigen-presenting cells (e.g., dendritic cells and macrophages) and monocytes in response to an infection with a variety of bacterial and fungal pathogens. The IL-23 receptor, IL-23R, is expressed on various adaptive and innate immune cells including Th17 cells, γδ T cells, natural killer (NK) cells, dendritic cells, macrophages, and innate lymphoid cells, which are found abundantly in the intestine. IL-23R and downstream effector cytokines have a key role in disease pathogenesis of inflammatory bowel disease (IBD) in acute and chronic mouse models. In patients with IBD, gene expression and protein levels of IL-23R are elevated at the intestine mucosal surface.

IL-23 inhibitors are an exciting new class of targeted molecules for treating IBD. For example, monoclonal antibodies directed to IL-23 have been shown to limit the differentiation of naïve T cells to T_{H}17 cells, thus ameliorating the pathogenesis of IBD (see, e.g., WO 2018/112232).

Despite the development of targeted IL-23 therapies (see Kashani and Schwartz (2019) Gastroenterol Hepatol NY 15(5):255), there remains an unmet medical need for alternative therapies that can mitigate disease burden, improve remission rates and modify progression in immune disorders such as IBD.

### SUMMARY OF THE INVENTION

In various aspects, the present disclosure provides a method of treating an autoimmune disorder and/or inflammatory bowel disease by administering a combination of an α4β7 inhibitor, such as an anti-α4β7 antibody, or an antigen-binding fragment thereof, and an IL-23 inhibitor, such as an anti-IL-23 antibody, or an antigen-binding fragment thereof.

In one aspect of the invention, provided herein is a method of treating a human patient in need thereof, said method comprising administering an α4β7 inhibitor and an IL-23 inhibitor to the human patient.

In one embodiment, the α4β7 inhibitor is an anti-α4β7 antibody. In one embodiment, the anti-α4β7 antibody is humanized. In one embodiment, the anti-α4β7 antibody comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

In one aspect of the invention, provided herein is a method of treating a human patient in need thereof, said method comprising administering an anti-α4β7 antibody and an IL-23 inhibitor to the human patient, wherein the anti-α4β7 antibody is an IgG1 antibody; comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

In one embodiment, the human patient has an autoimmune disease. In one embodiment, the autoimmune disease is psoriasis or arthritis. In certain embodiments, the autoimmune disease is rheumatoid arthritis, juvenile arthritis, psoriatic arthritis, or axial spondyloarthritis.

In one embodiment, the human patient has inflammatory bowel disease (IBD). In one embodiment, the IBD is ulcerative colitis (e.g., moderately to severely active ulcerative colitis) or Crohn's disease (e.g., moderately to severely active Crohn's disease).

In one embodiment, the anti-α4β7 antibody is administered before the IL-23 inhibitor.

In another embodiment, the anti-α4β7 antibody is administered after the IL-23 inhibitor.

In one embodiment, the anti-α4β7 antibody is administered concomitantly with the IL-23 inhibitor.

In another embodiment, the anti-α4β7 antibody comprises a heavy chain variable domain comprising an amino acid sequence as set forth in SEQ ID NO: 1, and comprises a light chain variable domain comprising an amino acid sequence as set forth in SEQ ID NO: 5.

In one embodiment, the anti-α4β7 antibody is a humanized antibody.

In one embodiment, the anti-α4β7 antibody is vedolizumab.

In one embodiment, the human patient is administered a first dose of 300 mg of the anti-α4β7 antibody at week 0, followed by a second dose of 300 mg of the anti-α4β7 antibody at week 2, followed by third dose of 300 mg of the anti-α4β7 antibody at week 6. In one embodiment, the human patient is administered 300 mg of the anti-α4β7 antibody every eight weeks beginning 8 weeks after the third dose. In a further embodiment, the human patient is administered 300 mg of the anti-α4β7 antibody every four weeks if the human patient is not showing clinical improvement.

In another embodiment, human patient is administered the human patient has ulcerative colitis or Crohn's disease and the clinical improvement is clinical remission.

In one embodiment, the human patient is administered 300 mg of the anti-α4β7 antibody every four weeks beginning 8 weeks after the third dose.

In an alternative embodiment, the human patient is administered 108 mg of the anti-α4β7 antibody to the human patient every two weeks beginning eight weeks after the third dose.

In one embodiment, the human patient is administered a first dose of 300 mg of the anti-α4β7 antibody at week 0, followed by a second dose of 300 mg of the anti-α4β7 antibody at week 2, followed by third dose of 108 mg of the anti-α4β7 antibody at week 6, followed by a 108 mg dose every two weeks thereafter.

In yet a further embodiment, the anti-α4β7 antibody is administered to the human patient intravenously. In certain embodiments, the 300 mg dose is administered intravenously.

In one embodiment, the anti-α4β7 antibody is administered to the human patient subcutaneously. In certain embodiments, the 108 mg dose is administered subcutaneously.

In one embodiment, the IL-23 inhibitor is an antibody that binds to the p19 subunit of IL-23.

In one embodiment, the IL-23 inhibitor is an antibody that binds to the p40 subunit of IL-23.

In one embodiment, the IL-23 inhibitor is an antibody that binds to the p19 and p40 subunits of IL-23.

In certain embodiments, the IL-23 inhibitor is an anti-IL-23 antibody, such as, but not limited to, risankizumab, ustekinumab, guselkumab, or tildrakizumab.

In one embodiment, the IL-23 inhibitor is an antibody that binds to IL-23R.

In one embodiment, the human patient has been characterized as a nonresponder or nonremitter at week 6 and/or week 10 after beginning treatment with an α4β7 inhibitor (e.g., an anti-α4β7 antibody, e.g., vedolizumab).

In one embodiment, the human patient has been characterized as having an elevated level of serum IL-22 at baseline or week 6 or week 10 after beginning treatment with an α4β7 inhibitor (e.g., an anti-α4β7 antibody, e.g., vedolizumab).

In one embodiment, serum IL-22 level of the human patient decreases none, less than two-fold, or less than three-fold from baseline to week 10 after beginning treatment with the anti-α4β7 antibody (e.g., vedolizumab), baseline to week 6 after beginning treatment with the anti-α4β7 antibody, or week 6 to week 10 after beginning treatment with the anti-α4β7 antibody.

In a further embodiment, the human patient has been characterized as having an elevated level of serum IL-22 at baseline or week 6 after beginning treatment with an anti-α4β7 antibody (e.g., vedolizumab), and wherein serum IL-22 level decreases none, less than two-fold, or less than three-fold from baseline to week 10, baseline to week 6 or week 6 to week 10.

In one embodiment, the human patient has been characterized as having an elevated level of serum IL-1β at baseline or week 6 or week 10 after beginning treatment with an α4β7 inhibitor (e.g., an anti-α4β7 antibody, e.g., vedolizumab).

In a further embodiment, the human patient has been characterized as having an elevated level of fecal calprotectin at baseline or week 6 after beginning treatment with an anti-α4β7 antibody (e.g., vedolizumab), and wherein the fecal calprotectin level decreases none, less than two-fold, or less than three-fold from baseline to week 10, baseline to week 6 or week 6 to week 10.

In certain embodiments, the human patient is characterized as having elevated levels of IL-22, STAT5A, and/or IL-1β as compared to a control level. In certain embodiments, the human patient is characterized as having elevated levels of IL-22, STAT5A, and/or IL-1β as compared to a reference level.

In one embodiment, the control level of IL-22, STAT5A, and/or IL-1β is a level from any one or more of a subject that does not suffer from IBD, a healthy subject, a non-inflamed colonic tissue, or a non-colonic tissue from the patient. In one embodiment, the reference level of IL-22 and/or STAT5A is a level determined from baseline levels of patients who responded to treatment with vedolizumab.

In one embodiment, the patient's IL-22, STAT5A, and/or IL-1β level is measured as a baseline, e.g., before treatment or on the first day of treatment with vedolizumab.

In another embodiment, the patient's IL-22, STAT5A, and/or IL-1β level, e.g., baseline level, is measured 1, 2, 3, 4, 5, 6, 7, 8, 9, and/or 10 days before treatment with vedolizumab.

In one embodiment, the patient's IL-22, STAT5A, and/or IL-1β nucleic acid and/or protein level is measured. In certain embodiments, the IL-22, STAT5A, and/or IL-1β level is elevated 5%, 10%, 15%, 20%, 25%, 30%, 40%, 50%, 60%, 70%, 80%, 90%, 100%, or more as compared to a control level or a reference level. In certain embodiments, the IL-22, STAT5A, and/or IL-1β level is elevated 5% to 35%, 5% to 25%, 5% to 20%, 5% to 15%, 5% to 10%, 10% to 20%, 10% to 15%, 10% to 30%, 15% to 40%, 20% to 50%, 25% to 60%, 30% to 70%, or 40% to 100%, or more as compared to a control level or a reference level.

In some aspects, the present invention provides a method of treating an inflammatory bowel disease in a patient in need thereof, comprising administering to the patient an anti-α4β7 antibody (e.g., vedolizumab) and an antibody that binds to the p19 subunit of IL-23, wherein the anti-α4β7 antibody is administered a first dose of 300 mg of the anti-α4β7 antibody at week 0, followed by a second dose of 300 mg of the anti-α4β7 antibody at week 2, a third dose of 300 mg of the anti-α4β7 antibody at week 6, followed by a 300 mg dose of the anti-α4β7 antibody to the human patient every eight weeks beginning 8 weeks after the third dose, wherein the anti-α4β7 antibody comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6, and wherein the patient is characterized as having elevated levels of IL-22, STAT5A, and/or IL-1β as compared to a control level.

In some aspects, the present invention provides a method of treating an inflammatory bowel disease in a patient in need thereof, comprising administering to the patient an anti-α4β7 antibody (e.g., vedolizumab) and an antibody that binds to the p40 subunit of IL-23, wherein the anti-α4β7 antibody is administered a first dose of 300 mg of the anti-α4β7 antibody at week 0, followed by a second dose of 300 mg of the anti-α4β7 antibody at week 2, a third dose of 300 mg of the anti-α4β7 antibody at week 6, followed by a 300 mg dose of the anti-α4β7 antibody to the human patient every eight weeks beginning 8 weeks after the third dose, wherein the anti-α4β7 antibody comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6, and wherein the patient is characterized as having elevated levels of IL-22, STAT5A, and/or IL-1β as compared to a control level.

In some aspects, the present invention provides a method of treating an inflammatory bowel disease in a patient in need thereof, comprising administering to the patient an anti-α4β7 antibody (e.g., vedolizumab) and an antibody that binds to the p19 subunit of IL-23, wherein the anti-α4β7 antibody is administered a first dose of 300 mg of the anti-α4β7 antibody at week 0, followed by a second dose of 300 mg of the anti-α4β7 antibody at week 2, followed by third dose of 108 mg of the anti-α4β7 antibody at week 6, followed by a 108 mg dose every two weeks thereafter, wherein the anti-α4β7 antibody comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6, and wherein the patient is characterized as having elevated levels of IL-22, STAT5A, and/or IL-1β as compared to a control level.

In other aspects, the present invention provides a method of treating an inflammatory bowel disease in a patient in need thereof, comprising administering to the patient an anti-α4β7 antibody (e.g., vedolizumab) and an antibody that binds to the p40 subunit of IL-23, wherein the anti-α4β7 antibody is administered a first dose of 300 mg of the anti-α4β7 antibody at week 0, followed by a second dose of 300 mg of the anti-α4β7 antibody at week 2, followed by third dose of 108 mg of the anti-α4β7 antibody at week 6, followed by a 108 mg dose every two weeks thereafter, wherein the anti-α4β7 antibody comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6, and wherein the patient is characterized as having elevated levels of IL-22, STAT5A, and/or IL-1β as compared to a control level.

In certain embodiments, the control level is a level from one or more of a subject that does not suffer from IBD, a healthy subject, a non-inflamed colonic tissue, or a non-colonic tissue from the patient

### DESCRIPTION OF FIGURES

FIG. 1. Baseline IL22 (1A) and STAT5A (1B) mRNA were higher in colon tissue from VDZ non-responder (VDZ-NR) UC patients as compared to responders (VDZ-R). mRNA expression was derived from microarray data generated in moderate/severe UC patients (Gene Expression Omnibus: GSE73661). Horizontal rectangle: Healthy controls (mean±1 SD).

FIG. 2: Baseline serum IL-22 levels were similar in VDZ remitters and non-remitters CD patients, but remained proportionally higher in non-remitters. Serum IL-22 was assessed in patients with moderate-to-severe CD. (A) IL-22 levels at baseline and week 10 in VDZ remitters and non-remitters. IL-22 levels were censored at the lower limit of quantification (2.7 pg/ml). (B) IL-22-fold change from baseline to week 10.

FIG. 3 provides a schematic of the study described in Example 1.

FIGS. 4A, 4B, and 4C graphically depict results from the mouse colitis study described in Example 1, where colon weight, diarrhea score, and histopathology score were quantified for healthy mice (normal), mice injected a negative IgG control, an anti-MADCAM antibody, an anti-p40 antibody, and both the anti-MADCAM antibody and the anti-p40 antibody.

FIG. 5A graphically depicts results from the study in Example 1 showing CD3% in the lamina propria (LP) and epithelium (EL) for the IgG injected group (negative control), the an anti-MADCAM antibody injected group, the anti-p40 antibody injected group, and the anti-MADCAM antibody and the anti-p40 antibody injected group. FIG. 5B shows a correlation between the CD3 percentage and total inflammatory cell infiltration.

FIGS. 6A and 6B graphically depict results showing levels of neutrophils (FIG. 6A) and macrophages (FIG. 6B) in the lamina propria at day 28 following administration of an IgG, an anti-MADCAM-1 antibody, an anti-p40 antibody, or the combination of an anti-MADCAM-1 antibody and an anti-p40 antibody.

FIG. 7 graphically depicts a diagram illustrating the synergistic effect of gene expression with the combination of anti-MADCAM-1 and anti-p40 therapy.

FIGS. 8A to 8C graphically depict results from administration of a vehicle control (negative control), an anti-MADCAM-1 antibody (MAdCAM-1), an anti-p40 antibody (p40), or a combination of the anti-MADCAM-1 antibody and the anti-p40 antibody (Combo) in a mouse model for colitis. Normal mice were used for comparison. FIG. 8A shows body weight (g) of the mice at day 21, FIG. 8B shows body weight of the mice at day 28, and FIG. 8C shows diarrhea score of the mice at day 21.

FIGS. 9A-9C graphically depict results from administration of a vehicle control (negative control), an anti-MADCAM-1 antibody (MAdCAM-1), an anti-p40 antibody (p40), or a combination of the anti-MADCAM-1 antibody and the anti-p40 antibody (Combo) in a mouse model for colitis. FIGS. 9A, 9B, and 9C describe levels of CD3(+) T cells, levels of MPO(+) neutrophils, and levels of CD68(+) macrophages, respectively, in the colon mucosa for each group tested and the normal control.

### DETAILED DESCRIPTION

### Definitions

In order that the present invention may be more readily understood, certain terms are first defined. In addition, it should be noted that whenever a value or range of values of a parameter are recited, it is intended that values and ranges intermediate to the recited values are also part of this invention.

The cell surface molecule, “α4β7 integrin,” or “α4β7” (used interchangeably throughout) is a heterodimer of an α4 chain (CD49D, ITGA4, OMIM 192975, human GeneID 3676) and a β7 chain (ITGB7, OMIM 147559; human GeneID 3695). Human α4-integrin and β7-integrin genes (GenBank (National Center for Biotechnology Information, Bethesda, Md.) RefSeq Accession numbers NM_000885 and NM_000889, respectively) are expressed by B and T lymphocytes, particularly memory CD4+ lymphocytes. Typical of many integrins, α4β7 can exist in either a resting or activated state. Ligands for α4β7 include vascular cell adhesion molecule (VCAM), fibronectin and mucosal addressin (MAdCAM (e.g., MAdCAM-1)).

As used herein, an “inhibitor of α4β7 integrin” or an “α4β7 inhibitor” inhibits the binding of α4β7 integrin to a ligand, e.g., MAdCAM, VCAM or fibronectin. An α4β7 inhibitor that inhibits the binding of α4β7 integrin with MAdCAM includes antibodies that bind an integrin protein complex comprising an α4 integrin, a β7 integrin, or α4 integrin and β7 integrin. Administering a polypeptide that inhibits MAdCAM-α4β7 integrin binding to inhibit α4β7 integrin activity is “anti-α4β7 integrin therapy.” In certain particular embodiments, a polypeptide that inhibits MAdCAM-α4β7 integrin binding is an anti-α4β7 integrin antibody, such as an antibody that will only bind α4 or β7 in the presence of the other, such as vedolizumab or a related antibody, or an antigen-binding fragment thereof. In some embodiments, an α4β7 inhibitor binds to the α4β7 integrin and blocks the interaction of α4β7 integrin with MAdCAM-1 and inhibits the migration of memory T-lymphocytes across the endothelium into inflamed gastrointestinal parenchymal tissue.

As used herein, an antibody, or antigen-binding fragment thereof, that has “binding specificity for the α4β7 complex” binds to α4β7, but not to α4β1 or αEB7. Vedolizumab is an example of an antibody that has binding specificity for the α4β7 complex.

As used herein, an “anti-α4β7 antibody” or “anti-α4β7 integrin antibody” refers to an antibody which specifically binds to α4β7 integrin. In one embodiment, an anti-α4β7 antibody blocks or inhibits the binding of α4β7 integrin to one or more of its ligands. In one embodiment, an anti-α4β7 antibody binds to α4β7, but not to α4β1 or αEB7. In one embodiment, an anti-α4β7 antibody is vedolizumab.

The term “antibody” broadly refers to an immunoglobulin molecule comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region (CH). The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

As used herein, the term “antibody fragment” or “antigen-binding fragment” of an antibody refers to Fab, Fab′, F(ab)₂, and Fv fragments, single chain antibodies, functional heavy chain antibodies (nanobodies), as well as any portion of an antibody having specificity toward at least one desired epitope, that competes with the intact antibody for specific binding (e.g., an isolated portion of a complementarity determining region having sufficient framework sequences so as to bind specifically to an epitope). Antigen binding fragments can be produced by recombinant techniques, or by enzymatic or chemical cleavage of an antibody.

As used herein, the term “humanized antibody” refers to an antibody that is derived from a non-human antibody (e.g., murine) that retains or substantially retains the antigen binding properties of the parent antibody but is less immunogenic in humans and contains minimal sequence derived from non-human immunoglobulins. Generally, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops (complementary determining regions) correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “IL-23 inhibitor” as used herein refers to an agent that inhibits or reduces IL-23 activity. The agent may, for example, bind IL-23 and/or IL-23R thereby inhibiting IL-23 activity. Alternatively, the agent may act to decrease levels of IL-23 mRNA or IL-23 protein. Examples of IL-23 inhibitors include, but are not limited to, antibodies or antigen-binding fragments thereof, small molecules, and nucleic acids (e.g., mRNA, DNA, siRNA, shRNA, antisense RNA, miRNA). An IL-23 inhibitor can act on either of the subunits of IL-23 (i.e., p19 or p40), or alternatively can act on both subunits, e.g., overlapping or combinatorial epitope. Thus, in some embodiments, an IL-23 inhibitor binds to p19 (e.g., anti-p19 antibody) and inhibits or reduces IL-23 activity. In other embodiments, an IL-23 inhibitor binds to p40 (e.g., anti-p40 antibody). Where the terms “p19” and “p40” are used herein, they refer to the respective subunits that make up human IL-23. In some embodiments, an IL-23 inhibitor binds to IL-23R (its subunits IL-23R and/or IL-12RB1). Thus, in some embodiments, the term “IL-23 inhibitor” encompasses an agent that binds to or inhibits any one or more of p19 (e.g., anti-p19 antibody), p40 (e.g., anti-p40 antibody), or IL-23R (anti-IL-23R antibody), and such terms are used interchangeably.

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

As used herein, the term “recombinant antibody” refers to an antibody produced as the result of the transcription and translation of a gene carried on a recombinant expression vector. In one embodiment, the vector has been introduced into a host cell. Alternatively, a vector can be used in a cell free system.

The term “baseline” refers to a starting point used for a comparison. In one embodiment, a baseline refers to a time point, e.g., day 0, prior to treatment with an anti-α4β7 antibody, or antigen binding fragment thereof.

The term “treatment” or “treating” means any treatment of a disease or disorder in a human subject, including: preventing or protecting against the disease or disorder, that is, causing the clinical symptoms not to develop; inhibiting the disease or disorder, that is, arresting or suppressing the development of clinical symptoms; and/or relieving the disease or disorder that is, causing the regression of clinical symptoms. In one embodiment, treatment of IBD is achieved where a subject having IBD sees an improvement in symptoms as measured by an accepted IBD index (e.g., a clinical measure for Crohn's disease or ulcerative colitis) dosing regimen with an anti-α4β7 antibody and an IL-23 inhibitor.

The term “therapeutically effective dose” is defined as an amount sufficient to cure or at least partially arrest the disease and its complications in a patient already suffering from the disease. In one embodiment, a therapeutically effective dose is a dose of an anti-α4β7 antibody and a dose of an IL-23 inhibitor that is able to improve a symptom and/or eliminate or reduce a complication (e.g., from prolonged steroid use) associated with IBD in a human subject having said disease. In one embodiment, a therapeutically effective dose is a dose of an anti-α4β7 antibody and a dose of an IL-23 inhibitor that is able to reduce a Crohn's Disease Activity Index (CDAI) score or reduce a modified CDAI to a score that is less than that which defined Crohn's disease in a human subject diagnosed with Crohn's disease. In one embodiment, a therapeutically effective dose is a dose of an anti-α4β7 antibody and a dose of an IL-23 inhibitor that is able to reduce a Mayo score or reduce to a score that is less than that which defined ulcerative colitis in a human subject diagnosed with ulcerative colitis.

As used herein, the term “clinical remission” as used herein with reference to ulcerative colitis subjects refers to a complete Mayo score of 2 or less points and no individual subscore greater than 1 point. Crohn's disease “clinical remission” refers to a CDAI score of 150 points or less.

The term “clinical response” as used herein with reference to ulcerative colitis subjects refers to a reduction in complete Mayo score of 3 or greater points and 30% from baseline, (or a partial Mayo score of 2 or greater points and 25% or greater from baseline, if the complete Mayo score was not performed at the visit) with an accompanying decrease in rectal bleeding subscore of 1 or greater points or absolute rectal bleeding score of 1 or less point. A “clinical response” as used herein with reference to Crohn's disease subjects refers to a 70 point or greater decrease in CDAI score from baseline (week 0).

As used herein, the terms “non-remitters,” “nonremitters” or “vedolizumab non-remitters” are used interchangeably to refer to a subset of patients having autoimmune disease and/or IBD (e.g., Crohn's disease or ulcerative colitis) who have received a first and second induction dose, or a third induction dose, of an α4β7 inhibitor, e.g., anti-α4β7 antibody (e.g., vedolizumab) and show signs of non-remission early in therapy, e.g., vedolizumab therapy (e.g., about three or four weeks after the second or third induction dose). For example, a patient may receive a dose of anti-α4β7 antibody (e.g., vedolizumab) at 0 and 2 weeks, or a dose at 0, 2 and 6 weeks. The patient may show clinical response to therapy, but not clinical remission, e.g., about three to four weeks after the second dose, e.g., at week 5 or 6, or about three to eight weeks after the third dose, e.g., at week 10 or week 14, after initiation of therapy with the anti-α4β7 antibody (e.g., vedolizumab). Signs of non-remission may include, e.g., inability to achieve clinical remission measures and/or measures described herein.

As used herein, the term “about” is used synonymously with the term “approximately.” Illustratively, the use of the term “about” indicates that values slightly outside the cited values, namely, plus or minus 5%.

### Therapeutic Uses and Methods

Provided herein are methods for treating a human patient in need thereof, using a combination therapy comprising an α4β7 inhibitor, e.g., an anti-α4β7 antibody and an IL-23 inhibitor. The combination therapy is based, at least in part, on the discovery described in the Examples below. For example, an anti-α4β7 antibody, such as vedolizumab, or an antigen-binding portion thereof, decreases inflammation associated with IBD, e.g., Crohn's disease, by blocking the trafficking of α4β7 T cells to the lamina propria of the colon, while an IL-23 inhibitor, such as an anti-IL23 antibody, or an antigen binding portion thereof, decreases inflammation in a human subject having Crohn's disease through the blockade of IL-23 derived from innate immune cells in the lamina propria.

By inhibiting or downregulation of the gut adaptive and innate immune system by vedolizumab and an IL-23 inhibitor, the two agents complement each other providing therapeutic improvements to intestinal inflammation in a subject having IBD.

By “combination therapy” in the context of the administration is intended to mean the use of two or more therapies, e.g., two or more agents, such as an α4β7 inhibitor, e.g., an anti-α4β7 antibody, or antigen-binding portion thereof, and an IL-23 inhibitor, e.g., an anti-p40, an anti-p19 antibody, or anti-IL-23R antibody, or antigen binding fragment thereof. The use of the term “in combination” or “combination therapy” does not restrict the order in which therapies are administered to a subject with a disease. A first therapy can be administered before (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks), concurrently, or after (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) the administration of a second therapy to a subject having a disease, such as inflammatory bowel disease (IBD). Any additional therapy can be administered in any order with the other additional therapies.

In one embodiment, the invention provides a method of treating a human patient in need thereof, said method comprising administering an α4β7 inhibitor, e.g., anti-α4β7 antibody, e.g., a humanized anti-α4β7 antibody (e.g., vedolizumab), or an antigen binding portion thereof, and an IL-23 inhibitor, (e.g., an anti-p40, an anti-p19 antibody, an anti-IL-23R antibody, or an antigen binding portion thereof), to the human patient. In certain embodiments, the human patient has an autoimmune disease, such as, but not limited to psoriasis or arthritis (e.g., rheumatoid arthritis, psoriatic arthritis, axial spondyloarthritis, juvenile arthritis), and/or inflammatory bowel disease (IBD), such as, but not limited to, ulcerative colitis or Crohn's disease. In certain embodiments, the human patient has moderately to severely active ulcerative colitis. In other embodiments, the human patient has moderately to severely active Crohn's disease.

In one embodiment, the α4β7 inhibitor, e.g., anti-α4β7 antibody (e.g., vedolizumab), or an antigen binding portion thereof, is administered before the IL-23 inhibitor, (e.g., an anti-p40, an anti-p19 antibody, an anti-IL-23R antibody, or an antigen binding portion thereof), for treatment. In other embodiments, the anti-α4β7 antibody (e.g., vedolizumab), or an antigen binding portion thereof, is administered after the IL-23 inhibitor, (e.g., an anti-p40, an anti-p19 antibody, or an anti-IL-23R antibody, or an antigen binding portion thereof). In yet other embodiments, the anti-α4β7 antibody (e.g., vedolizumab), or an antigen binding portion thereof, is administered concomitantly with the IL-23 inhibitor, (e.g., an anti-p40, an anti-p19 antibody, or an anti-IL-23R antibody, or an antigen binding portion thereof).

The methods disclosed herein include a combination therapy comprising administering both an α4β7 inhibitor, e.g., anti-α4β7 antibody (e.g., vedolizumab), or an antigen binding portion thereof, and an IL-23 inhibitor, (e.g., an anti-p40, an anti-p19 antibody, an anti-IL-23R antibody, or an antigen binding portion thereof). In some embodiments, an α4β7 inhibitor, e.g., the anti-α4β7 antibody (e.g., vedolizumab), or an antigen binding portion thereof, may be administered in accordance with its approved protocol and the IL23 inhibitor, (e.g., an anti-p40, an anti-p19 antibody, an anti-IL-23R antibody, or an antigen binding portion thereof), may be administered in accordance with its approved protocol.

In a combination therapy disclosed herein, an α4β7 inhibitor, e.g., an anti-α4β7 antibody (e.g., vedolizumab), or an antigen binding portion thereof, and an IL-23 inhibitor (e.g., an anti-p40 or an anti-p19 antibody, or an antigen binding portion thereof), may be administered by a variety of routes known in the art, such as orally, transdermally, subcutaneously, intranasally, intravenously, intramuscularly, intraocularly, or parenterally. The most suitable route for administration in any given case will depend on the particular α4β7 inhibitor, e.g., anti-α4β7 antibody and IL-23 inhibitor that is being administered, as well as, e.g., the patient, the formulation, the administration methods (e.g., administration time), the patient's age, body weight, body surface area, sex, severity of the diseases being treated, the patient's diet, and the patient's excretion rate.

In a combination therapy disclosed herein, an IL-23 inhibitor (e.g., an anti-IL23 antibody or anti-IL23R antibody) can be administered to a patient in need thereof (e.g., a patient with an autoimmune disease, such as arthritis or psoriasis, and/or inflammatory disease such as IBD) prior to administering an anti-α4β7 antibody (e.g., vedolizumab). In some embodiments, in a combination therapy disclosed herein, an IL-23 inhibitor can be administered to a patient 1 minute to 1 week (e.g., 1 minute, 5 minutes, 10 minutes, 15 minutes, 20 minutes, 25 minutes, 30 minutes, 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, or 7 days) or more prior to administering an α4β7 inhibitor, e.g., anti-α4β7 antibody (e.g., vedolizumab).

Alternatively, in a combination therapy disclosed herein, an IL-23 inhibitor (e.g., an anti-IL-23 antibody or anti-IL23 antibody) can be administered to a patient in need thereof (e.g., a patient with an autoimmune disease, such as arthritis or psoriasis, and/or inflammatory disease such as IBD) subsequent to administering an α4β7 inhibitor, e.g., anti-α4β7 antibody. In some embodiments, in a combination therapy disclosed herein, an IL-23 inhibitor can be administered to a patient 1 minute to 1 week (e.g., 1 minute, 5 minutes, 10 minutes, 15 minutes, 20 minutes, 25 minutes, 30 minutes, 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, or 7 days) or more subsequent to administering an α4β7 inhibitor, e.g., anti-α4β7 antibody (e.g., vedolizumab).

A combination therapy disclosed herein may be continued for any given time needed to treat the disease, e.g., IBD, including 1 week to 10 years (e.g., 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years) or more. For example, in a combination therapy disclosed herein, an α4β7 inhibitor, e.g., anti-α4β7 antibody (e.g., vedolizumab) and an IL-23 inhibitor (e.g., an anti-IL-23 antibody) can be administered for 1 week to 10 years (e.g., 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years) to a patient in need thereof (e.g., a patient with an autoimmune disease, such as arthritis or psoriasis and/or inflammatory disease such as IBD) in the doses and frequencies described herein.

In some embodiments, a patient with an autoimmune disease (e.g., arthritis or psoriasis—e.g., rheumatoid arthritis, juvenile arthritis, psoriatic arthritis, axial spondyloarthritis); or a chronic inflammatory disease (e.g., IBD) may continue to receive a combination therapy disclosed herein, until one or more symptoms of the disease is reduced (e.g., by 5% or more, such as by 5%, 10%, 15%, 20%, 25%, 30%, 35%, 40%, 45%, 50%, 55%, 60%, 65%, 70%, 75%, 80%, 85%, 90%, 95%, 97%, 99%, or more) in the patient. For example, a patient with IBD may continue to receive a combination of an α4β7 inhibitor, e.g., an anti-α4β7 antibody (e.g., vedolizumab) and an IL-23 inhibitor (e.g., an anti-IL-23 antibody or anti-IL-23R antibody) according to the methods described herein, until one or more symptoms of IBD is reduced (e.g., by 5% or more, such as by 5%, 10%, 15%, 20%, 25%, 30%, 35%, 40%, 45%, 50%, 55%, 60%, 65%, 70%, 75%, 80%, 85%, 90%, 95%, 97%, 99%, or more) in the patient relative to an initial baseline. For example, the IBD patient will have clinical remission or mucosal healing.

In one aspect, the invention provides a method of treating a disease or disorder in a subject comprising administering to a subject an α4β7 inhibitor, e.g., an anti-α4β7 antibody (e.g., vedolizumab) and an IL-23 inhibitor (e.g., an anti-IL-23 antibody or anti-IL-23R antibody) each in an amount effective to treat the disease or disorder, e.g., in humans. The human subject may be an adult (e.g., 18 years or older), an adolescent, or a child (juvenile or pediatric). The human subject may be a person 65 years or older. In certain embodiments, the human subject is a child who is less than 18 years old.

In certain embodiments, the subject is a responder to a treatment of a method according to an embodiment disclosed herein and is identified as having at least one of: (1) an endoscopic healing; (2) a clinical response; (3) a change (e.g., improvement) from baseline in Inflammatory Bowel Disease Questionnaire (IBDQ) score; (4) a mucosal healing; (5) a decrease from baseline in Mayo score; (6) a normalization of one or more biomarkers selected from the group consisting of C-reactive protein, fecal lactoferrin, albumin and fecal calprotectin; (7) a Psoriasis Area and Severity Index 75 (PASI) improvement relative to baseline; and (8) an improvement according to the American College of Rheumatology 20% response criteria (ACR20) or the 28-joint Disease Activity Score (DAS28).

In certain embodiments, the present invention provides a clinically proven safe and clinically proven effective method of treating moderately to severely active ulcerative colitis in a subject, wherein the subject is a responder to the treatment with the antibody combination and is identified as having a statistically significant improvement in disease activity as determined by endoscopic healing with a Mayo endoscopy subscore of 0 or 1 by a given time point, e.g., week 8, week 12, week 16, of treatment with the combination therapy.

In other embodiments, the present invention provides a clinically proven safe and clinically proven effective method of treating moderately to severely active ulcerative colitis in a subject, wherein the subject is a responder to the treatment with the α4β7 inhibitor, e.g., anti-α4β7 antibody (e.g., vedolizumab) and the IL-23 inhibitor (e.g., an anti-IL-23 antibody), and is identified as having a statistically significant improvement in disease activity as determined by an Ulcerative Colitis Endoscopic Index of Severity (UCEIS) score of less than or equal to 4 by a given time point, e.g., week 8, week 12, week 16, of treatment with the combination therapy.

In some embodiment, the present invention provides a method of treating an inflammatory disorder (e.g., moderately to severely active ulcerative colitis) in a subject, wherein the subject is a non-responder to the treatment with the α4β7 inhibitor, e.g., anti-α4β7 antibody (e.g., vedolizumab), by also treating with an IL-23 inhibitor, as described herein. In certain embodiments a non-responder or non-remitter is refractory to an α4β7 inhibitor, e.g., anti-α4β7 antibody treatment, and/or has elevated IL22 or STAT5, as described herein. Other signs of non-responsiveness may include clinical response measures and/or measures described herein. By way of example, a patient is characterized as a non-responder after the patient has received, e.g., at least two induction doses of an α4β7 inhibitor at 0 and 2 weeks, but after about 3-4 weeks, the patient shows no significant improvement of symptoms and/or shows clinical response without clinical remission. The non-responder patient continues to receive an α4β7 inhibitor treatment (e.g., vedolizumab 300 mg every 4 or 8 weeks or 108 mg every 2, 3 or 4 weeks), in combination with an IL-23 inhibitor. The combination therapy can last, e.g., for 4 to 20 weeks, 4 to 14 weeks, or 6 to 12 weeks. In some embodiments, the IL-23 inhibitor is administered until at least one genes selected from the group consisting of Itgal (αL chain/CD11a/LFA-1A), Itgb2 (β2 integrin chain/CD18), Itgax (αX chain/CD11c), Itga3, Itga9, Itgb1bk, Il21r, Il12rb1, Il12a, IL2ra, IL10ra, Il17re, Il34, Il18rap, Il1r11, Il1b, Il1r2, Il3ra, Il1f9, Il23a, Iltifb, Il6, Il18 bp, Il1a, Il15, Il1r1, Stat4, Stat2, and Cd3g is altered (e.g., upregulated or downregulated) as exemplified herein, and/or maintained until at least one of the genes is altered for at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, or more. In some embodiments, the IL-23 inhibitor is administered until a downregulation in integrin-beta-2 (Itgb2 (β2 integrin chain/CD18)) is achieved. In some embodiments, the IL-23 inhibitor is administered until a downregulation in IL1B (interleukin 1β) is achieved. In some embodiments, the IL-23 inhibitor is administered until a downregulation in IL23a is achieved. Downregulation can be, e.g., 5%, 10%, 15%, 20%, 25%, 30%, 35%, 40%, 45%, 50%, 55%, 60%, 65%, 70%, 75%, 80%, 85%, 90%, 95%, 100%, or more as compared to prior to IL-23 inhibitor combination therapy with an α4β7 inhibitor, e.g., anti-α4β7 antibody (e.g., vedolizumab). Downregulation can be 5% to 35%, 5% to 25%, 5% to 20%, 5% to 15%, 5% to 10%, 10% to 20%, 10% to 15%, 10% to 30%, 15% to 40%, 20% to 50%, 25% to 60%, 30% to 70%, or 40% to 100% or more as compared to prior to IL-23 inhibitor combination therapy with an α4β7 inhibitor, e.g., anti-α4β7 antibody (e.g., vedolizumab). In some embodiments, the patient receiving the combination therapy can return to a α4β7 inhibitor (e.g., anti-α4β7 antibody, e.g., vedolizumab) monotherapy when the patient has achieved remission.

In certain embodiments, the combination therapy described herein results in a human subject having IBD achieving a clinical response as defined herein for Crohn's disease or ulcerative colitis, e.g., as determined by a decrease from baseline in the Mayo score by greater than or equal to 30% and greater than or equal to 3 points and a decrease from baseline in the rectal bleeding subscore greater than or equal to 1 points or a rectal bleeding subscore of 0 or 1 by week 8 of treatment with the combination therapy.

In certain embodiments, the present invention provides a clinically proven safe and clinically proven effective method of treating IBD, e.g., moderately to severely active ulcerative colitis, or moderately to severely active Crohn's disease, in a human subject, wherein a subject identified as a non-responder to an initial treatment is administered a combination therapy described herein.

In certain embodiments, the combination therapy described herein results in a human subject having IBD achieving a clinical remission as defined herein for Crohn's disease or ulcerative colitis, wherein the subject identified as a non-remitter to an initial treatment is administered a combination therapy described herein.

In certain embodiments, the subject who is administered the combination therapy of the invention may have had a lack of an adequate response with, loss of response to, or was intolerant to treatment, e.g., initial treatment, with an immunomodulator, a TNF-alpha antagonist, or combinations thereof. In some embodiments, the subject who is administered the combination therapy of the invention may have had a lack of an adequate response or a lack of remission after initial treatment with an α4β7 inhibitor, e.g., anti-α4β7 antibody (e.g., vedolizumab). In certain embodiments, the subject who is administered the combination therapy of the invention may have had a lack of an adequate response with, loss of response to, or was dependent on corticosteroid therapy. The patient may have previously received treatment with at least one corticosteroid (e.g., prednisone) for the inflammatory bowel disease. An inadequate response to corticosteroids refers to signs and symptoms of persistently active disease despite a history of at least one 4-week induction regimen that included a dose equivalent to prednisone 30 mg daily orally for 2 weeks or intravenously for 1 week. A loss of response to corticosteroids refers to two failed attempts to taper corticosteroids to below a dose equivalent to prednisone 10 mg daily orally. Intolerance of corticosteroids includes a history of Cushing's syndrome, osteopenia/osteoporosis, hyperglycemia, insomnia and/or infection. A complication of prolonged steroid use can include venous thromboembolism, fragility fracture or infection.

An immunomodulator may be, for example, oral azathioprine, 6-mercaptopurine, or methotrexate. An inadequate response to an immunomodulator refers to signs and symptoms of persistently active disease despite a history of at least one 8-week regimen or oral azathioprine, 6-mercaptopurine, or methotrexate. Intolerance of an immunomodulator includes, but is not limited to, nausea/vomiting, abdominal pain, pancreatitis, LFT abnormalities, lymphopenia. TPMT genetic mutation and/or infection.

In one aspect, the subject may have had a lack of an adequate response with, loss of response to, or was intolerant to treatment a TNF-alpha antagonist. A TNF-alpha antagonist is, for example, an agent that inhibits the biological activity of TNF-alpha, and preferably binds TNF-alpha, such as a monoclonal antibody, e.g., REMICADE (infliximab), HUMIRA adalimumab), CIMZIA (certolizumab pegol. SIMPONI (golimumab) or an Fc fusion protein such as ENBREL (etanercept). An inadequate response to a TNF-alpha antagonist refers to signs and symptoms of persistently active disease despite a history of at least one 4-week induction regimen of infliximab 5 mg/kg IV, 2 doses at least 2 weeks apart; one 80 mg subcutaneous dose of adalimumab, followed by one 40 mg dose at least two weeks apart; or 400 mg subcutaneously of certolizumab pegol, 2 doses at least 2 weeks apart. A loss of response to a TNF-alpha antagonist refers to recurrence of symptoms during maintenance dosing following prior clinical benefit. Intolerance of a TNF-alpha antagonist includes, but is not limited to infusion related reaction, demyelination, congestive heart failure, and/or infection.

In one embodiment, diseases which can be treated accordingly with the combination therapy described herein include inflammatory bowel disease (IBD), such as ulcerative colitis, Crohn's disease, ileitis, Celiac disease, nontropical Sprue, enteropathy associated with seronegative arthropathies, microscopic or collagenous colitis, eosinophilic gastroenteritis, or pouchitis resulting after proctocolectomy, and ileoanal anastomosis. In one embodiment, the inflammatory bowel disease is Crohn's disease or ulcerative colitis. The ulcerative colitis may be moderate to severely active ulcerative colitis. Treatment may result in mucosal healing in patients suffering from moderate to severely active ulcerative colitis. Treatment may also result in a reduction, elimination, or reduction and elimination of corticosteroid use by the patient.

In other embodiments, the present invention also provides use of a combination of an α4β7 inhibitor and an IL-23 inhibitor for treating an autoimmune disease (e.g., arthritis or psoriasis, e.g., rheumatoid arthritis, juvenile arthritis, psoriatic arthritis, or axial spondyloarthritis) or an inflammatory bowel disease (e.g., ulcerative colitis or Crohn's disease, e.g., moderately to severely active ulcerative colitis; or moderately to severely active Crohn's disease), the use comprising administering an α4β7 inhibitor and an IL-23 inhibitor to a patient characterized as a nonresponder or a nonremitter to the α4β7 inhibitor, as described herein. In some embodiments, the α4β7 inhibitor is, e.g., an anti-α4β7 antibody, e.g., vedolizumab. In some embodiments, the vedolizumab is administered according to a dosing regimen as described herein.

In some embodiments, the patient has been characterized as a nonresponder or nonremitter at week 6 and/or week 10 after beginning treatment with an α4β7 inhibitor (e.g., an anti-α4β7 antibody, e.g., vedolizumab). In some embodiments, the patient has been characterized as having an elevated level of serum IL-22 at baseline or week 6 after beginning treatment with an α4β7 inhibitor (e.g., an anti-α4β7 antibody, e.g., vedolizumab). In some embodiments, the patient has been characterized as having an elevated level of serum IL-22 at baseline or week 6 after beginning treatment with vedolizumab, and wherein serum IL-22 level decreases none, less than two-fold, or less than three-fold from baseline to week 10, baseline to week 6 or week 6 to week 10. In some embodiments, the patient is characterized as having elevated levels of IL-22 and/or STAT5A as compared to a control level.

In some embodiments, the characterization occurs prior to treatment with the α4β7 inhibitor. In some embodiments, the characterization occurs after initial treatment with the α4β7 inhibitor. In one embodiment, IL-23 inhibitor is administered concomitantly with the α4β7 inhibitor. In one embodiment, the IL-23 inhibitor is administered after initial treatment with the α4β7 inhibitor. In some embodiments, the IL-23 inhibitor is administered for two weeks to 6 months, e.g., 2 to 4 weeks, 2 to 6 weeks, 2 to 8 weeks, 4 to 6 weeks, 4 to 8 weeks, 4 to 10 weeks, 1 to 2 months, 1 to 3 months, 2 to 3 months, 2 to 4 months, 3 to 4 months, 3 to 5 months, 4 to 5 months, 4 to 6 months, or 5 to 6 months. In some embodiments, the IL-23 inhibitor is administered until clinical remission is achieved. In certain embodiments, the IL-23 inhibitor is administered until one to 3 months (e.g., 4 to 6 weeks, 4 to 8 weeks, 4 to 10 weeks, 6 to 8 weeks, 6 to 10 weeks, 6 to 12 weeks, or 2 to 3 months) after clinical remission is achieved. In some embodiments, the IL-23 inhibitor is administered until at least one gene selected from certain integrin chain genes or certain cytokine genes. In some embodiments, the IL-23 inhibitor is administered until at least one gene selected from the group consisting of Itgal (αL chain/CD11a/LFA-1A), Itgb2 (β2 integrin chain/CD18), Itgax (αX chain/CD11c), Itga3, Itga9, Itgb1bk, Il21r, Il12rb1, Il12a, IL2ra, IL10ra, Il17re, Il34, Il18rap, Il1r11, Il1b, Il1r2, Il3ra, Il1f9, Il23a, Iltifb, Il6, Il18 bp, Il1a, Il15, Il1r1, Stat4, Stat2, and Cd3g is altered (e.g., upregulated or downregulated) as exemplified herein. In certain embodiments, administration of the α4β7 inhibitor (e.g., an anti-α4β7 antibody, e.g., vedolizumab) is continued after the IL-23 inhibitor is no longer administered.

Antibodies useful as anti-α4β7 antibodies or anti-IL-23 antibodies suitable in the methods and uses described herein can be identified using techniques known in the art, such as hybridoma production. Hybridomas can be prepared using, e.g., a murine system. Protocols for immunization and subsequent isolation of splenocytes for fusion are known in the art. Fusion partners and procedures for hybridoma generation are also known. In making a desired antibody, a target protein (antigen) of choice (whole protein or fragments thereof) is isolated and/or purified. Immunization of animals can be performed by any method known in the art. See, e.g., Harlow and Lane, Antibodies: A Laboratory Manual, New York: Cold Spring Harbor Press, 1990. Methods for immunizing animals such as mice, rats, sheep, goats, pigs, cattle and horses are well known in the art. See, e.g., Harlow and Lane, supra, and U.S. Pat. No. 5,994,619. A desired antigen may be administered with an adjuvant to stimulate the immune response. Adjuvants known in the art include complete or incomplete Freund's adjuvant, RIBI (muramyl dipeptides) or ISCOM (immunostimulating complexes). After immunization of an animal with a desired antigen, antibody-producing immortalized cell lines are prepared from cells isolated from the immunized animal. After immunization, the animal is sacrificed and lymph node and/or splenic B cells are immortalized by methods known in the art (e.g., oncogene transfer, oncogenic virus transduction, exposure to carcinogenic or mutating compounds, fusion with an immortalized cell, e.g., a myeloma cell, and inactivating a tumor suppressor gene. See, e.g., Harlow and Lane, supra. Hybridomas can be selected, cloned and further screened for desirable characteristics, including robust growth, high antibody production and desirable antibody characteristics. Human anti-PCDH17 antibodies can also be generated in mice, such as in the HuMAb-Mouse® or XenoMouse™.

Methods for high throughput screening of antibody, or antibody fragment, libraries for molecules capable of binding a target protein (antigen) can be used to identify and affinity mature antibodies useful for the methods of the present disclosure. Such methods include in vitro display techniques known in the art, such as phage display, bacterial display, yeast display, mammalian cell display, ribosome display, mRNA display, and cDNA display, among others. The use of phage display to isolate ligands that bind biologically relevant molecules has been reviewed, for example, in Felici et al., Biotechnol. Annual Rev. 1:149-183, 1995; Katz, Annual Rev. Biophys. Biomol. Struct. 26:27-45, 1997; and Hoogenboom et al., Immunotechnology 4:1-20, 1998, the disclosures of each of which are incorporated herein by reference as they pertain to in vitro display techniques. Randomized combinatorial peptide libraries have been constructed to select for polypeptides that bind cell surface antigens as described in Kay, Perspect. Drug Discovery Des. 2:251-268, 1995 and Kay et al., Mol. Divers. 1:139-140, 1996, the disclosures of each of which are incorporated herein by reference as they pertain to the discovery of antigen-binding molecules. Proteins, such as multimeric proteins, have been successfully phage-displayed as functional molecules (see, for example, EP 0349578; EP 4527839; and EP 0589877, as well as Chiswell and McCafferty, Trends Biotechnol. 10:80-84 1992, the disclosures of each of which are incorporated herein by reference as they pertain to the use of in vitro display techniques for the discovery of antigen-binding molecules). In addition, functional antibody fragments, such as Fab and scFv fragments, have been expressed in in vitro display formats (see, for example, McCafferty et al., Nature 348:552-554, 1990; Barbas et al., Proc. Natl. Acad. Sci. USA 88:7978-7982, 1991; and Clackson et al., Nature 352:624-628, 1991, the disclosures of each of which are incorporated herein by reference as they pertain to in vitro display platforms for the discovery of antigen-binding molecules). These techniques, among others, can be used to identify and improve the affinity of antibodies that bind to a target antigen.

In addition to in vitro display techniques, computational modeling techniques can be used to design and identify antibodies, or antibody fragments, in silico that bind a target antigen. For example, using computational modeling techniques, one of skill in the art can screen libraries of antibodies, or antibody fragments, in silico for molecules capable of binding specific epitopes, such as extracellular epitopes of the target antigen.

Examples of α4β7 inhibitor, e.g., anti-α4β7 antibodies, or fragments thereof, and IL-23 inhibitors that may be used in the methods disclosed herein are provided below.

### α4β7 Inhibitors

The methods disclosed herein comprise administering both an α4β7 inhibitor, e.g., anti-α4β7 antibody, or an antigen-binding portion thereof, and an IL-23 inhibitor, e.g., an anti-IL-23 antibody, to a subject having an autoimmune disease, such as arthritis or psoriasis (e.g., rheumatoid arthritis, juvenile arthritis, psoriatic arthritis, axial spondyloarthritis), and/or inflammatory disease such as IBD (e.g., Crohn's disease or ulcerative colitis) for treatment.

A variety of polypeptides can inhibit α4β7 integrin binding to MAdCAM, including: anti-α4β7 antibodies as described herein, anti-MAdCAM antibodies, soluble integrin subunits (including fusion proteins, such as Fc-fusions), and soluble MAdCAM (including fusion proteins, such as Fc-fusions). Primate MAdCAMs are described in, e.g., PCT publication WO 96/24673, the entire teachings of which are incorporated herein by this reference. Polypeptides that inhibit MAdCAM-α4β7 integrin binding and can be used consonant with the invention include: anti-MAdCAM antibodies (see, e.g., U.S. Pat. No. 8,277,808, PF-00547659, SHP647, ontamalimab or antibodies described in WO2005/067620); soluble integrin subunits (e.g., complexes comprising α4 and/or β7, which lack a transmembrane domain or lack a transmembrane and intracellular domains) such as soluble α4 integrin, soluble β7 integrin, or soluble α4β7 integrin complex, including fusion proteins, such as Fc fusions, comprising soluble integrin subunits; and soluble MAdCAM (e.g., lacking a transmembrane domain or lacking a transmembrane and intracellular domains), including fusion proteins comprising MAdCAM, such as MAdCAM-Fc chimera, as described in, for example, U.S. Pat. No. 7,803,904.

The present invention relies, at least in part, on an anti-α4β7 antibody, or an antigen-binding portion thereof, which (1) can bind −α4β7 integrin in vitro and/or in vivo: and (2) can modulate an activity or function of α4β7 integrin, such as (a) binding function (e.g., the ability of α4β7 integrin to bind to MAdCAM (e.g., MAdCAM-1), fibronectin and/or VCAM-1) and/or (b) leukocyte infiltration function, including recruitment and/or accumulation of leukocytes in tissues (e.g., the ability to inhibit lymphocyte migration to intestinal mucosal. tissue). In one embodiment, an antibody used herein can bind an α4β7 integrin, and can inhibit binding of the α4β7 integrin to one or more of its ligands (e.g. MAdCAM (e.g., MAdCAM-1), VCAM-1, fibronectin), thereby inhibiting leukocyte infiltration of tissues (including, recruitment and/or accumulation of leukocytes in tissues). In another embodiment, an anti-α4β7 antibody used herein can bind α4β7 integrin, and can selectively inhibit binding of the α4β7 integrin to one or more of its ligands (e.g., MAdCAM (e.g., MAdCAM-1), VCAM-1, fibronectin), thereby inhibiting leukocyte infiltration of tissues (including recruitment and/or accumulation of leukocytes in tissues). Such anti-α4β7 antibodies can inhibit cellular adhesion of cells bearing an α4β7 integrin to vascular endothelial cells ire mucosal tissues, including gut-associated tissues, lymphoid organs or leukocytes (especially lymphocytes such as T or B cells) in vitro and/or in vivo. In yet another embodiment, the anti-α4β7 antibody used herein can inhibit the interaction of α4β7 with MAdCAM (e.g., MAdCAM-1) and/or fibronectin. In still yet another embodiment, the anti-α4β7 antibody used herein can inhibit the interaction of α4β7 with MAdCAM (e.g., MAdCAM-1) and/or fibronectin selectively, e.g., without inhibiting the interaction of α4β7 with VCAM.

Thus, the anti-α4β7 antibody used in the methods disclosed herein, can be used to modulate (e.g., inhibit (reduce or prevent)) binding function and/or leukocyte (e.g., lymphocyte, monocyte) infiltration function of α4β7 integrin. For example, humanized immunoglobulins which inhibit the binding of α4β7 integrin to a ligand (i.e., one or more ligands) can be administered according to the method in the treatment of diseases associated with leukocyte (e.g., lymphocyte, monocyte) infiltration of tissues (including recruitment and/or accumulation of leukocytes in tissues), particularly of tissues which express the molecule MAdCAM (e.g., MAdCAM-1).

Anti-α4β7 antibodies for use in the methods provided herein can, in certain embodiments, bind to an epitope on the α4 chain, on the β7 chain, or to a combinatorial epitope formed by the association of the α4 chain with the β7 chain. In one aspect, the antibody is specific for the α4β7 integrin complex, e.g., the antibody binds a combinatorial epitope on the α4β7 complex, but does not bind an epitope on the α4 chain or the β7 chain unless the chains are in association with each other. In another aspect, the anti-α4β7 antibody binds both the α4 integrin chain and the β7 integrin chain, and thus, is specific for the α4β7 integrin complex. Such antibodies, which are specific for the α4β7 integrin complex, can bind α4β7 but not bind α4β1, and/or not bind α_{E}β7, for example. In another aspect, the anti-α4β7 antibody binds to the same or substantially the same epitope as the Act-1 antibody (Lazarovits, A. I. et al., J. Immunol., 133(4): 1857-1862 (1984), Schweighoffer et al., J. Immunol., 151(2): 717-729, 1993; Bednarczyk et al., J. Biol. Chem., 269(11): 8348-8354, 1994). Murine ACT-1 Hybridoma cell line, which produces the murine Act-1 monoclonal antibody, was deposited under the provisions of the Budapest Treaty on Aug. 22, 2001, on behalf of Millennium Pharmaceuticals, Inc., 40 Landsdowne Street, Cambridge, Mass. 02139, U.S.A., at the American Type Culture Collection, 10801 University Boulevard, Manassas, Va. 20110-2209, U.S.A., under Accession No. PTA-3663. In another aspect, the anti-α4β7 antibody is a human antibody or an α4β7 binding protein using the CDRs provided in U.S. Patent Application Publication No. 2010/0254975. In other embodiments, the anti-α4β7 integrin antibody is AMG181 (abrilumab, specific for α4β7, see, e.g., U.S. Pat. No. 8,444,981), etrolizumab (β7-specific, FIB504 or a humanized derivative (e.g., Fong et al., U.S. Pat. No. 7,528,236), CAS 1044758-60-2, KEGG D09901, PubChem 124490613; see, e.g., U.S. Pat. No. 7,528,236), natalizumab (α4-specific, humanized MAb 21.6, TYSABRI®, CAS 189261-10-7, KEGG D06886, PubChem 49661786; see, e.g., U.S. Pat. No. 5,840,299), a related antibody of any of the foregoing, an antigen-binding fragment of any of the foregoing, or a combination thereof. Treatment methods using anti-α4β7 integrin antibodies are described in publication nos. U.S. 2005/0095238, WO2012151248 and WO 2012/151247.

An effective amount of an α4β7 inhibitor, e.g., anti-α4β7 antibody is administered to a human subject in order to treat such a disease. For example, inflammatory diseases, including diseases which are associated with leukocyte infiltration of the gastrointestinal tract (including gut-associated endothelium), other mucosal tissues, or tissues expressing the molecule MAdCAM (e.g. MAdCAM-1) (e.g., gut-associated tissues, such as venules of the lamina propria of the small and large intestine; and mammary gland (e.g., lactating mammary gland)), can be treated according to the present method. Similarly, an individual having a disease associated with leukocyte infiltration of tissues as a result of binding of leukocytes to cells (e.g., endothelial cells) expressing MAdCAM (e.g., MAdCAM-1) can be treated according to the present invention.

In certain embodiments, the α4β7 inhibitor, e.g., anti-α4β7 antibody, or an antigen-binding fragment thereof, is a humanized anti-α4β7 antibody, or an antigen-binding fragment thereof, that is an IgG1 antibody; comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

In certain embodiments, the anti-α4β7 antibody, or an antigen-binding fragment thereof, is a humanized anti-α4β7 antibody comprising a heavy chain variable domain comprising an amino acid sequence as set forth in SEQ ID NO: 1, and comprising a light chain variable domain comprising an amino acid sequence as set forth in SEQ ID NO: 5.

In certain embodiments, the anti-α4β7 antibody is vedolizumab.

In some embodiments where the anti-α4β7 antibody, e.g., humanized anti-α4β7 antibody is vedolizumab or an antibody having vedolizumab binding regions, the human patient is administered a first dose of 300 mg of the anti-α4β7 antibody, e.g., humanized anti-α4β7 antibody at week 0, followed by a second dose of 300 mg of the anti-α4β7 antibody, e.g., humanized anti-α4β7 antibody at week 2, followed by third dose of 300 mg of the anti-α4β7 antibody, e.g., humanized anti-α4β7 antibody at week 6. The method may further comprise administering 300 mg of the anti-α4β7 antibody, e.g., humanized anti-α4β7 antibody to the human patient every eight weeks beginning 8 weeks after the third dose. In an alternative embodiment, the method described herein may comprise administering 300 mg of the anti-α4β7 antibody, e.g., humanized anti-α4β7 antibody to the human patient every four weeks if the human patient is not showing clinical improvement, e.g., clinical remission of Crohn's disease or ulcerative colitis. In other embodiments, the method comprises administering, e.g., intravenously, 300 mg of the anti-α4β7 antibody, e.g., humanized anti-α4β7 antibody to the human patient every four weeks beginning 8 weeks after the third dose. In a further embodiment, in certain instances the method comprises administering 108 mg of the anti-α4β7 antibody, e.g., humanized anti-α4β7 antibody to the human patient every two weeks beginning eight weeks after the third dose. In certain embodiments, the method comprises administering a first dose of 300 mg of the anti-α4β7 antibody, e.g., humanized anti-α4β7 antibody at week 0, followed by a second dose of 300 mg of the anti-α4β7 antibody, e.g., humanized anti-α4β7 antibody at week 2, followed by administering a third dose of 108 mg of the anti-α4β7 antibody, e.g., humanized anti-α4β7 antibody at week 6, followed by a 108 mg dose every two weeks thereafter. In certain embodiments, the 108 mg dose is administered subcutaneously. In certain embodiments, the 108 mg dose is self-administered.

In some embodiments where the anti-α4β7 antibody, e.g., humanized anti-α4β7 antibody is vedolizumab or an antibody having vedolizumab binding regions, a juvenile (pediatric) human patient can be treated according to the methods provided herein. For example, a juvenile human patient is administered a first dose of 200 mg of the anti-α4β7 antibody, e.g., humanized anti-α4β7 antibody, a second dose of 200 mg of the antibody two weeks after the first dose, and a third dose of 200 mg of the antibody six weeks after the first dose. The method may further comprise administering a fourth dose of 200 mg at 14 weeks after the first dose. The method may also further comprise administering subsequent doses of 200 mg every eight weeks thereafter. In an alternative embodiment, the juvenile human patient is administered a first dose of 100 mg of the anti-α4β7 antibody, e.g., humanized anti-α4β7 antibody, a second dose of 100 mg of the antibody two weeks after the first dose, and a third dose of 100 mg of the antibody six weeks after the first dose. The method may further comprise administering a fourth dose of 200 mg at 14 weeks after the first dose. The method may also further comprise administering a fifth and subsequent dose of 200 mg every eight weeks after the fourth dose. In another embodiment, the juvenile human patient is administered a first dose of 150 mg of an anti-α4β7 antibody, e.g., humanized anti-α4β7 antibody, a second dose of 150 mg of the antibody two weeks after the first dose, and a third dose of 150 mg of the antibody six weeks after the first dose. The method further comprises a fourth dose of 150 mg at 14 weeks after the first dose. The method may also further comprise a fourth dose of 300 mg at 14 weeks after the first dose. The method may also further comprise a fifth and subsequent dose of 150 mg or 300 mg every eight weeks after the fourth dose. In certain embodiments, a juvenile human patient is administered a first dose of 300 mg of an anti-α4β7 antibody, e.g., humanized anti-α4β7 antibody, a second dose of 300 mg of the antibody two weeks after the first dose, and a third dose of 300 mg of the antibody six weeks after the first dose. The method further comprises a fourth dose of 300 mg at 14 weeks after the first dose. The method may also further comprise a fifth and subsequent dose of 300 mg every eight weeks after the fourth dose. In certain embodiments, the juvenile human patient is administered a first dose of 200 mg of an anti-α4β7 antibody, e.g., humanized anti-α4β7 antibody, a second dose of 200 mg of the antibody two weeks after the first dose, and subcutaneously administering a third dose of 108 mg of the antibody six weeks after the first dose and subsequent doses of 108 tug of the antibody every two, three or four weeks thereafter. In some embodiments, a pediatric patient under 30 kg in weight is administered a higher dose than a pediatric patient over 30 kg. Other specific embodiments are described, e.g., in PCT publication WO 2018/200818, incorporated by reference in its entirety.

In particular, the methods disclosed herein include administration of anti-α4β7 antibody vedolizumab, or antibodies having antigen binding regions of vedolizumab. Vedolizumab is also known by its trade name ENTYVIO® (Takeda Pharmaceuticals, Inc.). Vedolizumab is a humanized antibody that comprises mutated human IgG1 framework regions and antigen-binding CDRs from the murine antibody Act-1 (which is described in U.S. Pat. No. 7,147,851, incorporated by reference herein).

Vedolizumab specifically binds to the α4β7 integrin and blocks the interaction of α4β7 integrin with mucosal addressin cell adhesion molecule-1 (MAdCAM-1) and fibronectin and inhibits the migration of memory T-lymphocytes across the endothelium into inflamed gastrointestinal parenchymal tissue. Vedolizumab does not bind to or inhibit function of the α4β1 and αEβ7 integrins and does not antagonize the interaction of α4 integrins with vascular cell adhesion molecule-1 (VCAM-1).

The α4β7 integrin is expressed on the surface of a discrete subset of memory T-lymphocytes that preferentially migrate into the gastrointestinal tract. MAdCAM-1 is expressed on gut endothelial cells and plays a critical role in the homing of T-lymphocytes to gut lymph tissue. The interaction of the α4β7 integrin with MAdCAM-1 has been implicated as an important contributor to mucosal inflammation, such as the chronic inflammation that is a hallmark of ulcerative colitis and Crohn's disease. Vedolizumab may be used to treat inflammatory bowel disease, including Crohn's disease and ulcerative colitis, HIV, pouchitis, including chronic pouchitis, fistulizing Crohn's disease, graft versus host disease and celiac disease. These diseases are contemplated for the combination therapies described herein.

The heavy chain variable region of vedolizumab is provided herein as SEQ ID NO: 1, and the light chain variable region of vedolizumab is provided herein as SEQ ID NO: 5. Vedolizumab comprises a heavy chain variable region comprising a CDR1 of SEQ ID NO: 2, a CDR2 of SEQ ID NO: 3, and a CDR3 of SEQ ID NO: 4. Vedolizumab comprises a light chain variable region comprising a CDR1 of SEQ ID NO: 6, a CDR2 of SEQ ID NO: 7 and CDR3 of SEQ ID NO: 8. Vedolizumab and the sequences of vedolizumab are also described in U.S. Patent Publication No. 2014/0341885 and U.S. Patent Publication No. 2014/0377251, the entire contents of each which are expressly incorporated herein by reference in their entireties. Alpha4beta7 antibodies and their corresponding amino acid sequences are described in U.S. patent Ser. No. 10/143,752, which is incorporated by reference herein.

Also included in the invention is the use of the methods disclosed herein with an alternative antibody. Specifically, the methods described herein may be performed using an antibody, or an antigen binding fragment thereof, that binds to α4 integrin, including, but not limited to natalizumab. In another alternative, the methods described herein may be performed using an antibody, or an antigen binding fragment thereof, that binds to β7 integrin, including, but not limited to etrolizumab which binds to both integrins α4β7 and αEβ7 (sequences of etrolizumab are described in US 20180086833, which is incorporated by reference herein).

### IL-23 Inhibitors

As described above, the methods disclosed herein comprise administering both an α4β7 inhibitor, e.g., anti-α4β7 antibody, or an antigen-binding portion thereof, and an IL-23 inhibitor, e.g., an anti-IL-23 antibody or an anti-IL-23R antibody, to a subject having an autoimmune disease, such as arthritis or psoriasis, and/or an inflammatory disease such as IBD (e.g., Crohn's disease or ulcerative colitis) for treatment.

The term “human interleukin-23” or “hIL-23” as used herein refers to a heterodimeric pro-inflammatory cytokine formed by an IL12B subunit (RefSeq Accession number NM_002187.3; NP_002178.2) and an IL23A subunit (RefSeq Accession number NM_016584.3; NP_057668.1). “IL12B” is also known as p40, IL-12p40, CLFM2, and NKSF2; this p40 subunit is also a subunit that forms IL-12. The amino acid sequence of p40 is described in SEQ ID NO: 13. “IL23A” is also known as p19, IL-23p19, IL-23 subunit alpha, and SGRF. The amino acid sequence of p19 is described in SEQ ID NO: 14. IL-23 binds to its receptor, IL-23R, which comprises two subunits IL23R (RefSeq Accession number NM_144701.3; NP_653302.2) and IL-12RB1 (RefSeq Accession number NM_005535.3; NP_005526.1). Both subunits are required for IL-23 signaling.

In certain embodiments, an IL-23 inhibitor, which can be used in the combination therapy disclosed herein, is an anti-IL-23 antibody or an antigen-binding fragment thereof, that specifically binds to human IL-23, either the p19 subunit, the p40 subunit, or a shared epitope thereof. In certain embodiments, an IL-23 inhibitor, which can be used in the combination therapy disclosed herein, is an anti-IL-23R antibody or an antigen-binding fragment thereof, that specifically binds to human IL-23R.

In one embodiment, an anti-IL-23 antibody that can be used in the combination therapy disclosed herein is brazikumab. Brazikumab is also known as AMG 139, MEDI2070, or MEDI-2070. Brazikumab is known in the art, including, for example, as described in U.S. Pat. Nos. 8,722,033 and 9,487,580, which are incorporated by reference in their entireties with respect to brazikumab, including the sequences thereof.

In some embodiments, an anti-IL-23 antibody (e.g., an anti-p19 antibody) suitable for use in the combination therapy disclosed herein is guselkumab. Guselkumab is also known as TREMFYA™ or CNTO 1959. Guselkumab is known in the art, including, for example, as described in U.S. Pat. Nos. U.S. Pat. No. 7,491,391 and U.S. Ser. No. 10/030,070, which are incorporated by reference in their entireties with respect to guselkumab, including the sequences thereof.

In some embodiments, an anti-IL-23 antibody (e.g., an anti-p19 antibody) suitable for use in the combination therapy disclosed herein is risankizumab. Risankizumab is also known as SKYRIZI™, BI 655066, or ABBV-066. Risankizumab is known in the art, including, for example, as described in U.S. Pat. Nos. U.S. Pat. Nos. 8,778,346 and 9,441,036, which are incorporated by reference in their entireties with respect to risankizumab, including the sequences thereof.

In some embodiments, an anti-IL-23 antibody (e.g., an anti-p40 antibody) suitable for use in the combination therapy disclosed herein is briakinumab. Briakinumab is also known as ABT-874, J695, BSF 415977, LU 415977, A-796874.0, and WAY-165772. Briakinumab is described in, e.g., U.S. Pat. Nos. 6,914,128, 7,504,485, 9,072,725, and US20150037348, incorporated by reference in their entireties with respect to briakinumab, including the sequences thereof.

In some embodiments, an anti-IL-23 antibody (e.g., an anti-p19 antibody) suitable for use in the combination therapy disclosed herein is tildrakizumab. Tildrakizumab is also known as ILUMYA™, tildrakizumab-asmn, MK-3222, SCH 900222, or hum13B8-b. Tildrakizumab is known in the art, including, for example, as described in U.S. Pat. Nos. U.S. Pat. Nos. 8,362,212, 8,404,813 and 9,803,010, which are incorporated by reference in their entireties with respect to tildrakizumab, including the sequences thereof.

In some embodiments, an anti-IL-23 antibody (e.g., an anti-p40 antibody) that can be used in the combination therapy disclosed herein is ustekinumab. Ustekinumab is also known as CNTO 1275, TT-20, C01275, or STELARA™. Ustekinumab is known in the art, e.g., U.S. Pat. No. 6,902,734, WO2002012500, U.S. Pat. No. 7,279,157, US 20200095315, incorporated by reference in their entireties with respect to the ustekinumab, including sequences thereof.

Another example of an anti-IL-23 antibody that can be used in the combination therapy disclosed herein is LY2525623. LY2525623 is known in the art, including, for example, as described in U.S. Pat. Nos. U.S. Pat. Nos. 7,872,102 and 9,023,358, which are incorporated by reference in their entireties with respect to LY2525623, including the sequences thereof.

In one embodiment, an anti-IL-23 antibody that can be used in the combination therapy disclosed herein is CNTO 4088. CNTO 4088 is known in the art, including, for example, as described in U.S. Pat. Nos. U.S. Pat. Nos. 7,807,414 and 7,935,344, which are incorporated by reference in their entireties with respect to CNTO 4088.

In some embodiments, an anti-IL-23 antibody that can be used in the combination therapy disclosed herein is mirikizumab. Mirikizumab is also known as LY3074828. Mirikizumab is described in, e.g., Ma et al. (Expert Opin Investig Drugs, 27:649-660 (2018)), and Reich et al. (Br J Dermatol, 181:88-95 (2019)), incorporated by reference herein in their entireties with respect to mirikizumab.

Other anti-IL-23 antibodies suitable for use in the combination therapy disclosed herein are known in the art, including, for example, as described in U. S. Application Nos. US20110206686 and US20120264917, U.S. Pat. Nos. U.S. Pat. Nos. 9,127,057 and 7,510,709, and in Li et al. (Anal Chem 89:2250-2258 (2017)), which are incorporated by reference in their entireties with respect to IL-23 antibodies. Other anti-IL-23 antibodies (e.g., an anti-p19 antibody) suitable for use in the combination therapy disclosed herein are described in, e.g., WO2007147019, WO2008134659, WO2009082624, U.S. Pat. No. 8,333,968, US20110159589, US20120294862, US20130309235, U.S. Pat. No. 9,464,134, and US20150197566, incorporated by reference in their entireties with respect to IL-23 antibodies.

Other anti-IL-23 antibodies (e.g., an anti-p40 antibody) suitable for use in the combination therapy disclosed herein are described in, e.g., US20150010544, U.S. Pat. No. 9,708,395, and US20160333085, incorporated by reference in their entireties with respect to IL-23 antibodies.

In certain embodiments, an IL-23 inhibitor suitable for use in the methods described herein is a small molecule inhibitor of IL-23. In some embodiments, a small molecule inhibitor of IL-23 may reduce or inhibit the production of IL-23, such as by reducing or inhibiting IL-23 production at the transcription or translation level. In some embodiments, a small molecule inhibitor of IL-23 may reduce or inhibit the function of IL-23, e.g., by reducing or inhibiting the interaction of IL-23 with its receptor (IL-23R), and/or reducing or inhibiting the ability of the subunits p19 and p40 to form a complex.

In some embodiments, a small molecule inhibitor of IL-23 suitable for use in the combination therapy disclosed herein is STA-5326. STA-5326 inhibits the production of IL-23 through the prevention of nuclear translocation of c-Rel. STA-5326 is also known as Apilimod mesylate, Apilimod, LAM-002. The chemical name of STA-5326 is (E)-4-(6-(2-(3-methylbenzylidene)hydrazinyl)-2-(2-(pyridin-2-yl)ethoxy)pyrimidin-4-yl)morpholine dimethanesulfonate. STA-5326 is described in, e.g., Burakoff et al. (Inflamm Bowel Dis, 12:558-565 (2006)) and Wada et al. (Blood, 109:1156-1164 (2007)) incorporated by reference in its entirety.

In some embodiments, an anti-IL-23R antibody suitable for use in the combination therapy disclosed herein is AS2762900-00. AS2762900-00 is described in, e.g., U.S. Pat. No. 9,556,276, US20140275490, WO2012137676, U.S. Pat. No. 9,371,391, US20150126713, WO2013129454, Imamura et al. (Eur J Pharmacol, 824:163-169 (2018)), Sasaki-Iwaoka et al. (Eur J Pharmacol, 828:89-96 (2018)), and Sasaki-Iwaoka et al. (Eur J Pharmacol, 843:190-198 (2019)), incorporated by reference in their entireties with respect to IL-23R antibodies.

Other examples of anti-IL-23R antibodies suitable for use in the combination therapy disclosed herein are described in, e.g., US20100166767, WO2008106134, US20110158992, WO2010027767, US20120148582, U.S. Pat. No. 8,691,532, and US20140170154, incorporated by reference in their entireties with respect to IL-23R antibodies.

### Patient Selection

In some embodiments, a patient is selected for treatment with the combination therapy disclosed herein based on certain characteristics. In some embodiments, the patient is a nonresponder to α4β7 inhibitor therapy. As used herein, “non-responders,” “nonresponders” or “vedolizumab non-responders” are a subset of patients having autoimmune disease and/or IBD (e.g., Crohn's disease or ulcerative colitis) who have received a first and second induction dose of an α4β7 inhibitor, e.g., anti-α4β7 antibody (e.g., vedolizumab) and show signs of non-responsiveness early in therapy, e.g., vedolizumab therapy (e.g., about three or four weeks after the second induction dose). Signs of non-responsiveness may include, e.g., inability to achieve clinical response measures and/or measures described herein. In some embodiments, the patient is a nonremitter to α4β7 inhibitor therapy. As used herein, “non-remitters,” “nonremitters” or “vedolizumab non-remitters” are a subset of patients having autoimmune disease and/or IBD (e.g., Crohn's disease or ulcerative colitis) who have received a first and second induction dose, or a third induction dose, of an α4β7 inhibitor, e.g., anti-α4β7 antibody (e.g., vedolizumab) and show signs of non-remission early in therapy, e.g., vedolizumab therapy (e.g., about three or four weeks after the second or third induction dose). For example, a patient may receive a dose of anti-α4β7 antibody (e.g., vedolizumab) at 0 and 2 weeks, or a dose at 0, 2 and 6 weeks. The patient may show clinical response to therapy, but not clinical remission, e.g., about three to four weeks after the second dose e.g., at week 5 or 6, or about three to eight weeks after the third dose, e.g., at week 10 or week 14, after initiation of therapy with the anti-α4β7 antibody (e.g., vedolizumab). Signs of non-remission may include, e.g., inability to achieve clinical remission measures and/or measures described herein.

In some embodiments, non-responsiveness may be identified using an algorithm comprising factors including, but not limited to, α4β7 inhibitor, e.g., anti-α4β7 antibody concentration and/or antibody clearance. Antibody concentration may be measured in serum obtained from the patient. In further embodiments, factors in the algorithm for identifying treatment for non-responsiveness may comprise body weight and/or albumin levels. Nonresponders to α4β7 inhibitor therapy may have, e.g., high inhibitor clearance, low albumin levels, high fecal calprotectin amounts, a lack of reduction in fecal calprotectin amounts after initial treatment, and/or high body weight.

In some embodiments, the patient is characterized as having elevated levels of a marker gene, e.g., IL-22, STAT5A, and/or IL-1β. In some embodiments, the level of IL-22, STAT5A, and/or IL-1β is elevated 5%, 10%, 15%, 20%, 25%, 30%, 35%, 40%, 45%, 50%, 55%, 60%, 65%, 70%, 75%, 80%, 85%, 90%, 95%, 100%, or more or in certain embodiments, the IL-22, STAT5A, and/or IL-1r3 level is elevated 5% to 35%, 5% to 25%, 5% to 20%, 5% to 15%, 5% to 10%, 10% to 20%, 10% to 15%, 10% to 30%, 15% to 40%, 20% to 50%, 25% to 60%, 30% to 70%, or 40% to 100%, or more as compared to control levels, e.g., from a non-IBD subject, such as in a control sample from a healthy human or control non-inflamed colonic tissue or non-colonic tissue having been obtained from the patient, or as compared to a reference level in responders to the α4β7 inhibitor. The elevated levels may be measured in a colonic tissue biopsy having been obtained from the patient. The biopsy sample may be obtained before treatment (e.g., 2 to 10 days before) or on the day before or on the first day of treatment with α4β7 inhibitor, e.g., anti-α4β7 antibody, e.g., vedolizumab. The measurement may test the level of nucleic acid, e.g., on a microarray, or protein, e.g., by immunohistochemistry using methods well known in the art.

In some embodiments, IL-22 (interleukin-22) has the Gene ID 50616, at least 95%, 97%, 99% or 100% identical to SEQ ID NO: 9 (GenBank Accession number NM_020525.5) or the protein of SEQ ID NO: 10 (expressed as GenPept Accession number NP_065386.1) in the databases maintained by the National Center for Biotechnology Information, Bethesda, Md. In some embodiments, STAT5A (signal transducer and activator of transcription 5A) has the Gene ID 6776, at least 95%, 97%, 99% or 100% identical to SEQ ID NO: 11 (GenBank Accession number NM_003152.3) or the protein of SEQ ID NO: 12 (expressed as GenPept Accession number NP_003143.2) in the databases maintained by the National Center for Biotechnology Information, Bethesda, Md. In some embodiments, IL-13 (interleukin-1-beta) has the Gene ID 3553, at least 95%, 97%, 99% or 100% identical to GenBank Accession number NM_000576) or the protein of expressed as GenPept Accession number NP_000567) in the databases maintained by the National Center for Biotechnology Information, Bethesda, Md.

In some embodiments, the patient to be administered combination therapy is characterized as having an elevated level of serum IL-22 at baseline, week 6 or week 10 after beginning α4β7 inhibitor, e.g., anti-α4β7 antibody, e.g., vedolizumab treatment. In some embodiments, the patient to be administered combination therapy is characterized as having an elevated level of serum IL-22 at baseline or week 6 after beginning α4β7 inhibitor, e.g., anti-α4β7 antibody, e.g., vedolizumab treatment that decreases none, less than two-fold or less than three-fold from baseline to week 10, baseline to week 6 or week 6 to week 10. In some embodiments, an elevated level of serum IL-22 is >3 pg/ml, 5 to 100 pg/ml, >5 pg/ml, >10 pg/ml, 10 to 120 pg/ml, >10 pg/ml or more. In some embodiments, in remitters, the level of serum IL-22 reduces to less than 10 pg/ml, less than 5 pg/ml, less than 3 pg/ml, less than 2.7 pg/ml, less than 2 pg/ml, or is undetectable, after initial therapy, e.g., by week 6 or week 10 of treatment. In some embodiments, in nonremitters, the level of serum IL-22 does not reduce to less than 10 pg/ml, less than 5 pg/ml, less than 3 pg/ml, less than 2.7 pg/ml, less than 2 pg/ml, or is not undetectable, after initial therapy, e.g., by week 6 and/or week 10 of treatment.

In some embodiments, the patient to be administered combination therapy is characterized as having an elevated level of fecal calprotectin at baseline, week 6, or week 10 after beginning α4β7 inhibitor, e.g., anti-α4β7 antibody, e.g., vedolizumab treatment. In some embodiments, the patient to be administered combination therapy is characterized as having an elevated level of fecal calprotectin at baseline or week 6 after beginning α4β7 inhibitor, e.g., anti-α4β7 antibody, e.g., vedolizumab treatment that decreases none, less than two-fold or less than three-fold from baseline to week 10, baseline to week 6, or week 6 to week 10. See, e.g., US2017360926, incorporated by reference in its entirety. In some embodiments, the patient to be administered combination therapy is characterized as having an elevated level of fecal calprotectin that is 5%, 10%, 15%, 20%, 25%, 30%, 35%, 40%, 45%, 50%, 55%, 60%, 65%, 70%, 75%, 80%, 85%, 90%, 95%, 100%, or more, or 5% to 35%, 5% to 25%, 5% to 20%, 5% to 15%, 5% to 10%, 10% to 20%, 10% to 15%, 10% to 30%, 15% to 40%, 20% to 50%, 25% to 60%, 30% to 70%, 40% to 100%, or more as compared to normal level of fecal calprotectin.

as compared to normal levels. or in certain embodiments, the IL-22, STAT5A, and/or IL-1β level is elevated In some embodiments, the patient to be administered combination therapy is characterized as having an elevated level of both IL-22 and fecal calprotectin at baseline, week 6 or week 10 after beginning α4β7 inhibitor, e.g., anti-α4β7 antibody, e.g., vedolizumab treatment. In some embodiments, the patient to be administered combination therapy is characterized as having an elevated level of both IL-22 and fecal calprotectin at baseline or week 6 after beginning α4β7 inhibitor, e.g., anti-α4β7 antibody, e.g., vedolizumab treatment that decreases none, less than two-fold or less than three-fold from baseline to week 10, baseline to week 6, or week 6 to week 10.

In some embodiments, the patient to be administered combination therapy is a nonresponder, e.g., does not show clinical response to vedolizumab at week 6 and/or week 10 after beginning vedolizumab treatment. In some embodiments, the patient to be administered combination therapy is a nonremitter, e.g., does not show clinical remission to vedolizumab at week 6 and/or week 10 after beginning vedolizumab treatment.

The term “sample”, as used herein, is intended to include a sample, e.g., tissue, cells, biological fluids and isolates thereof, isolated from a subject, as well as tissues, cells and fluids present within a subject and can be obtained from a patient or a normal subject. A noninvasive sample, e.g., for in vitro measurement of markers to identify responders or nonresponders, can include blood or serum sample. Accordingly, a blood sample can be tested for marker characteristic, e.g., size, sequence, composition, activity or amount (i.e., level). For patients with an autoimmune disease or IBD, a control, reference sample for normal marker characteristic, e.g., size, sequence, composition, activity or amount (i.e., level) can be obtained from a healthy subject not having IBD.

Blood collection containers can comprise an anti-coagulant, e.g., heparin or ethylene-diaminetetraacetic acid (EDTA), sodium citrate or citrate solutions with additives to preserve blood integrity, such as dextrose or albumin or buffers, e.g., phosphate. If the amount of marker is being measured by measuring the level of its DNA in the sample, a DNA stabilizer, e.g., an agent that inhibits DNAse, can be added to the sample. If the amount of marker is being measured by measuring the level of its RNA in the sample, an RNA stabilizer, e.g., an agent that inhibits RNAse, can be added to the sample. If the amount of marker is being measured by measuring the level of its protein in the sample, a protein stabilizer, e.g., an agent that inhibits proteases, can be added to the sample. An example of a blood collection container is PAXGENE™ tubes (PREANALYTIX, Valencia, Calif.), useful for RNA stabilization upon blood collection. Peripheral blood samples can be modified, e.g., fractionated, sorted or concentrated (e.g., for a reference sample).

The sample, e.g., blood or modified blood, and/or the reference, e.g., matched control (e.g., germline), sample can be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the characteristic e.g., size, sequence, composition, activity or amount (i.e., level) of the marker gene (e.g., IL-22, STAT5A, and/or IL-1β) in the sample.

In some embodiments, a marker can be identified by sequencing a nucleic acid marker, e.g., a DNA, RNA, cDNA or a protein marker correlated with the marker gene, e.g., IL-22, STAT5A, and/or IL-1β. There are several sequencing methods known in the art to sequence nucleic acids. A primer or primer pair can be used for sequencing one or both strands of DNA corresponding to the marker gene. A primer can be used in conjunction with a probe, e.g., a nucleic acid probe, e.g., a hybridization probe, to amplify a region of interest prior to sequencing to boost sequence amounts for detection of a marker gene. Examples of regions which can be sequenced include an entire gene, transcripts of the gene and a fragment of the gene or the transcript, e.g., one or more of exons or untranslated regions or a portion of a marker comprising a mutation site. Examples of sequences to target for primer selection and sequence or composition analysis can be found in public databases which collect sequence or mutation information, such as RefSeq, COSMIC and dbGaP.

Sequencing methods are known to one skilled in the art. Examples of methods include the Sanger method, the SEQUENOM™ method and Next Generation Sequencing (NGS) methods. The Sanger method, comprising using electrophoresis, e.g., capillary electrophoresis to separate primer-elongated labeled DNA fragments, can be automated for high-throughput applications. The primer extension sequencing can be performed after PCR amplification of regions of interest.

In some embodiments, DNA marker, e.g., genomic DNA of the marker gene (e.g., IL-22, STAT5A, and/or IL-1β) can be analyzed both by in situ and by in vitro formats in a biological sample using methods known in the art. DNA can be directly isolated from the sample or isolated after isolating another cellular component, e.g., RNA or protein. Kits are available for DNA isolation, e.g., QIAAMP™ DNA Micro Kit (Qiagen, Valencia, Calif.). DNA also can be amplified using such kits.

In another embodiment, mRNA marker of the marker gene (e.g., IL-22, STAT5A, and/or IL-1β) can be analyzed both by in situ and by in vitro formats in a biological sample using methods known in the art. Many expression detection methods use isolated RNA. For in vitro methods, any RNA isolation technique that does not select against the isolation of mRNA can be utilized for the purification of RNA from cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155). RNA can be isolated using standard procedures (see e.g., Chomczynski and Sacchi (1987) Anal. Biochem. 162:156-159), solutions (e.g., trizol, TRI REAGENT™ (Molecular Research Center, Inc., Cincinnati, Ohio; see U.S. Pat. No. 5,346,994) or kits (e.g., a QIAGEN™ Group RNEASY® isolation kit (Valencia, Calif.) or LEUKOLOCK™ Total RNA Isolation System, Ambion division of Applied Biosystems, Austin, Tex.).

Additional steps may be employed to remove DNA from RNA samples. Cell lysis can be accomplished with a nonionic detergent, followed by microcentrifugation to remove the nuclei and hence the bulk of the cellular DNA. DNA subsequently can be isolated from the nuclei for DNA analysis. In one embodiment, RNA is extracted from cells of the various types of interest using guanidinium thiocyanate lysis followed by CsCl centrifugation to separate the RNA from DNA (Chirgwin et al. (1979) Biochemistry 18:5294-99). Poly(A)+RNA is selected by selection with oligo-dT cellulose (see Sambrook et al. (1989) Molecular Cloning—A Laboratory Manual (2nd ed.), Cold Spring Harbor Laboratory, Cold Spring Harbor, N.Y.). Alternatively, separation of RNA from DNA can be accomplished by organic extraction, for example, with hot phenol or phenol/chloroform/isoamyl alcohol. If desired, RNAse inhibitors may be added to the lysis buffer. Likewise, for certain cell types, it may be desirable to add a protein denaturation/digestion step to the protocol. For many applications, it is desirable to enrich mRNA with respect to other cellular RNAs, such as transfer RNA (tRNA) and ribosomal RNA (rRNA). Most mRNAs contain a poly(A) tail at their 3′ end. This allows them to be enriched by affinity chromatography, for example, using oligo(dT) or poly(U) coupled to a solid support, such as cellulose or SEPHADEX™ medium (see Ausubel et al. (1994) Current Protocols In Molecular Biology, vol. 2, Current Protocols Publishing, New York). Once bound, poly(A)+mRNA is eluted from the affinity column using 2 mM EDTA/0.1% SDS.

Analyzing a characteristic of a marker gene described herein in a biological sample involves obtaining a biological sample (e.g., a blood sample or a reference sample) from a test subject. The characteristic can be assessed by any of a wide variety of well known methods for detecting or measuring the characteristic, e.g., of a marker or plurality of markers, e.g., of a nucleic acid (e.g., RNA, mRNA, genomic DNA, or cDNA) and/or translated protein. Non-limiting examples of such methods include immunological methods for detection of secreted, cell-surface, cytoplasmic, or nuclear proteins, protein purification methods, protein function or activity assays, nucleic acid hybridization methods, optionally including “mismatch cleavage” steps (Myers, et al. (1985) Science 230:1242) to digest mismatched, i.e., mutant or variant, regions and separation and identification of the mutant or variant from the resulting digested fragments, nucleic acid reverse transcription methods, and nucleic acid amplification methods and analysis of amplified products. These methods include gene array/chip technology, RT-PCR, TAQMAN™ gene expression assays (Applied Biosystems, Foster City, Calif.), e.g., under GLP approved laboratory conditions, in situ hybridization, immunohistochemistry, immunoblotting, FISH (fluorescence in situ hybridization), FACS analyses, northern blot, southern blot, INFINIUM™ DNA analysis Bead Chips (Illumina, Inc., San Diego, Calif.), quantitative PCR, bacterial artificial chromosome arrays, single nucleotide polymorphism (SNP) arrays (Affymetrix, Santa Clara, Calif.) or cytogenetic analyses.

Detection methods can be used to detect RNA, mRNA, protein, cDNA, or genomic DNA, for example, in a biological sample in vitro as well as in vivo. Furthermore, in vivo techniques for detection of a polypeptide or nucleic acid marker described herein include introducing into a subject a labeled probe to detect the marker, e.g., a nucleic acid complementary to the transcript of a marker or a labeled antibody, Fc receptor or antigen directed against the polypeptide, e.g., wild type or mutant marker. For example, the antibody can be labeled with a radioactive isotope whose presence and location in a subject can be detected by standard imaging techniques. These assays can be conducted in a variety of ways. A skilled artisan can select from these or other appropriate and available methods based on the nature of the marker(s), tissue sample and mutation in question. Some methods are described in more detail in later sections. Different methods or combinations of methods could be appropriate in different cases or, for instance in different patient populations.

In vitro techniques for detection of a polypeptide corresponding to a marker of the invention include enzyme linked immunosorbent assays (ELISAs), Western blots, protein array, immunoprecipitations, immunochemistry and immunofluorescence. In such examples, expression of a marker is assessed using an antibody (e.g., an unlabeled, a radio-labeled, chromophore-labeled, fluorophore-labeled, or enzyme-labeled antibody), an antibody derivative (e.g., an antibody conjugated with a substrate or with the protein or ligand of a protein-ligand pair (e.g., biotin-streptavidin)), or an antibody fragment (e.g., a single-chain antibody, an isolated antibody hypervariable domain, etc.) which binds specifically with a marker protein or fragment thereof, e.g., a protein or fragment comprising a region which can be mutated or a portion comprising a mutated sequence, or a mutated residue in its structural context, including a marker protein which has undergone all or a portion of its normal post-translational modification. An antibody can detect a protein with an amino acid sequence selected from the group consisting of SEQ ID NO:10 and 12. An assay such as a sandwich ELISA assay could detect a gain or loss of quantity of the marker in the sample, e.g., in comparison to the reference sample, or a standard ELISA would compare the levels of binding of the antibodies between patient and reference sample.

In some embodiments, the method includes measuring the amount (i.e., level) of marker protein. In some embodiments, the amount of marker protein is quantified by immunohistochemistry of a sample, e.g. using Ventana Medical Systems (Arizona) or 11lumina (San Diego) quantitation products and methods. In some embodiments, the amount of marker protein is quantified by immunohistochemistry of a marker from blood. In some embodiments, the amount of marker protein is determined by a score of antibody binding or staining intensity. In some embodiments, the amount of marker protein is determined by comparison of the antibody binding or staining in a healthy cell or tissue versus a cell or tissue from a subject having an autoimmune disease or IBD.

In one embodiment, expression of a marker is assessed by preparing mRNA/cDNA (i.e., a transcribed polynucleotide) from cells in a patient sample, and by hybridizing the mRNA/cDNA with a reference polynucleotide which is a complement of a marker nucleic acid, or a fragment thereof. cDNA can, optionally, be amplified using any of a variety of polymerase chain reaction methods prior to hybridization with the reference polynucleotide. Expression of one or more markers likewise can be detected using quantitative PCR to assess the level of expression of the marker(s). An example of the use of measuring mRNA levels is that an inactivating mutation in a marker gene can result in an altered level of mRNA in a cell. The level can be upregulated due to feedback signaling protein production in view of nonfunctional or absent protein or downregulated due to instability of an altered mRNA sequence. Alternatively, any of the many known methods of detecting mutations or variants (e.g. single nucleotide polymorphisms, deletions, etc., discussed above) of a marker of the invention may be used to detect occurrence of a mutation in a marker gene in a patient.

An example of direct measurement is quantification of transcripts. As used herein, the level or amount of expression refers to the absolute amount of expression of an mRNA encoded by the marker or the absolute amount of expression of the protein encoded by the marker. As an alternative to making determinations based on the absolute expression amount of selected markers, determinations may be based on normalized expression amounts. Expression amount can be normalized by correcting the absolute expression level of a marker upon comparing its expression to the expression of a control marker that is not a marker, e.g., in a housekeeping role that is constitutively expressed. Suitable markers for normalization also include housekeeping genes, such as the actin gene or beta-2 microglobulin. Reference markers for data normalization purposes include markers which are ubiquitously expressed and/or whose expression is not regulated by oncogenes or cytokines. Constitutively expressed genes are known in the art and can be identified and selected according to the relevant tissue and/or situation of the patient and the analysis methods. Such normalization allows one to compare the expression level in one sample, to another sample, e.g., between samples from different times or different subjects. Further, the expression level can be provided as a relative expression level. The baseline of a genomic DNA sample, e.g., diploid copy number, can be determined by measuring amounts in cells from subjects not having an autoimmune disorder or IBD. To determine a relative amount of a marker or marker set, the amount of the marker or marker set is determined for at least 1, or 2, 3, 4, 5, or more samples, e.g., 7, 10, 15, 20 or 50 or more samples in order to establish a baseline, prior to the determination of the expression level for the sample in question. To establish a baseline measurement, the mean amount or level of each of the markers or marker sets assayed in the larger number of samples is determined and this is used as a baseline expression level for the biomarkers or biomarker sets in question. The amount of the marker or marker set determined for the test sample (e.g., absolute level of expression) is then divided by the baseline value obtained for that marker or marker set. This provides a relative amount and aids in identifying abnormal levels of marker protein activity.

Probes based on the sequence of a nucleic acid molecule of the invention can be used to detect transcripts or genomic sequences corresponding to one or more markers of the invention. The probe can comprise a label group attached thereto, e.g., a radioisotope, a fluorescent compound, an enzyme, or an enzyme co-factor. Such probes can be used as part of a diagnostic test kit for identifying cells or tissues which express the protein, such as by measuring levels of a nucleic acid molecule encoding the protein in a sample of cells from a subject, e.g., detecting mRNA levels or determining whether a gene encoding the protein has been mutated or deleted.

Primers or nucleic acid probes comprise a nucleotide sequence complementary to a specific a marker and are of sufficient length to selectively hybridize with a marker gene or nucleic acid associated with a marker gene. Primers and probes can be used to aid in the isolation and sequencing of marker nucleic acids. In one embodiment, the primer or nucleic acid probe, e.g., a substantially purified oligonucleotide, comprises a region having a nucleotide sequence which hybridizes under stringent conditions to about (plus or minus 5% within the value of) 6, 8, 10, 12, or 15, 20, 25, 30, 40, 50, 60, 75, 100 or more consecutive nucleotides of a marker gene. In another embodiment, the primer or nucleic acid probe is capable of hybridizing to a marker nucleic acid comprising a nucleotide sequence of any sequence set forth in any of SEQ ID NOs: 9 or 11. For example, a primer or nucleic acid probe comprising a nucleotide sequence of at least about 15 consecutive nucleotides, at least about 25 nucleotides or having from about 15 to about 20 nucleotides set forth in any of SEQ ID NOs: 9 or 11. Nucleic acid analogs can be used as binding sites for hybridization. An example of a suitable nucleic acid analogue is peptide nucleic acid (see, e.g., Egholm et al., Nature 363:566 568 (1993); U.S. Pat. No. 5,539,083).

Primers or nucleic acid probes can be selected using an algorithm that takes into account binding energies, base composition, sequence complexity, cross-hybridization binding energies, and secondary structure (see Friend et al., International Patent Publication WO 01/05935, published Jan. 25, 2001; Hughes et al., Nat. Biotech. 19:342-7 (2001). One of skill in the art can design primers and nucleic acid probes for the markers disclosed herein or related markers with similar characteristics, e.g., markers on the chromosome loci, or mutations in different regions of the same marker gene described herein, using the skill in the art, e.g., adjusting the potential for primer or nucleic acid probe binding to standard sequences, mutants or allelic variants by manipulating degeneracy or GC content in the primer or nucleic acid probe. Computer programs that are well known in the art are useful in the design of primers with the required specificity and optimal amplification properties, such as Oligo version 5.0 (National Biosciences, Plymouth, Minn.). While perfectly complementary nucleic acid probes and primers can be used for detecting the markers described herein and mutants, polymorphisms or alleles thereof, departures from complete complementarity are contemplated where such departures do not prevent the molecule from specifically hybridizing to the target region. For example, an oligonucleotide primer may have a non-complementary fragment at its 5′ end, with the remainder of the primer being complementary to the target region. Alternatively, non-complementary nucleotides may be interspersed into the nucleic acid probe or primer as long as the resulting probe or primer is still capable of specifically hybridizing to the target region.

The following examples exemplify improved methods and compositions described herein. The following examples are offered for illustrative purposes only, and are not intended to limit the scope of the present invention in any way. Commercially available reagents referred to in the examples were used according to manufacturer's instructions unless otherwise indicated.

### EXAMPLES

### Example 1. Efficacy of Anti-MAdCAM-1 Antibody and Anti-p40 Antibody Combination in Mouse Colitis Model

The following study evaluates the combined therapeutic benefit of a combination therapy including an anti-MAdCAM-1 antibody and an IL-23 inhibitor, specifically antibody that bound and blocked the p40 subunit of IL-23 (and IL-12) using a murine colitis model. As described below, the combination of the two therapeutic agents (anti-MAdCAM-1 (MECA-367 from BioXCell (Cat #BE0035)) and anti-p40 IL-23/IL-12 (clone C17.8 from BioXCell (Cat #BE0051)) resulted in improvements in the colon weight, diarrhea, and histopathology in the tested mice vs. controls.

The model used was a naïve T cell transfer (TCT) model where CD4+ adoptive transfer colitis is mainly driven by Th1/Th17-mediated immune response. Disease in the mice is characterized by infiltration of lamina propria with CD4+ T cells, neutrophils and macrophages, resulting in progressive weight loss, colonic inflammation, and diarrhea.

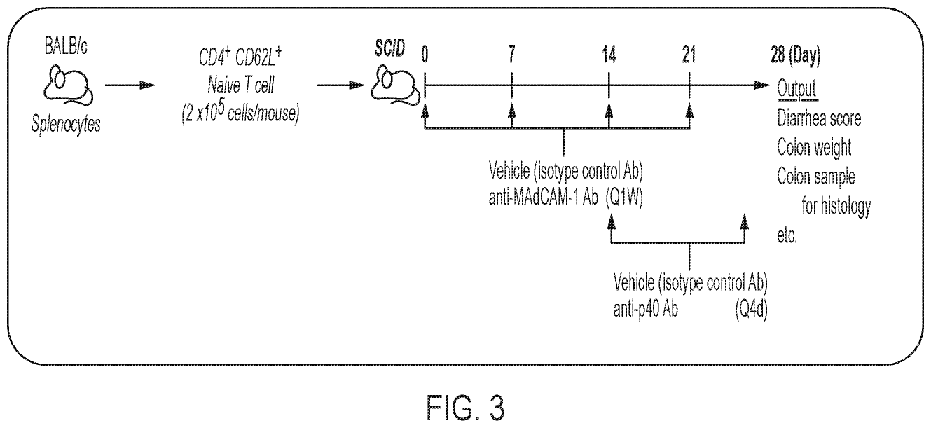

A schematic overview of the study is described in FIG. 3. Briefly, naïve T cells (CD4+CD62L+) from BALB/c mice were transferred to SCID mice (approximately 2×10⁵cells/mouse). Mice were treated with isotype control antibody (“vehicle”), 10 mg/kg anti-MAdCAM antibody, 1 mg/kg anti-p40 IL12/IL23 antibody (anti-p40 antibody), or a combination of 10 mg/kg anti-MAdCAM antibody and 1 mg/kg anti-p40 IL12/IL23 antibody.

As shown in FIG. 3, vehicle or 10 mg/kg anti-MAdCAM antibody were administered at Day 0, Day 7, Day 14, Day 21, and Day 28 (Q1W). Vehicle or 1 mg/kg anti-p40 IL12/IL23 antibody were administered once every four days (Q4d) starting at Day 14 and ending at Day 26. Samples were collected for analysis, e.g., diarrhea score, colon weight, colon histology, colon mRNA.

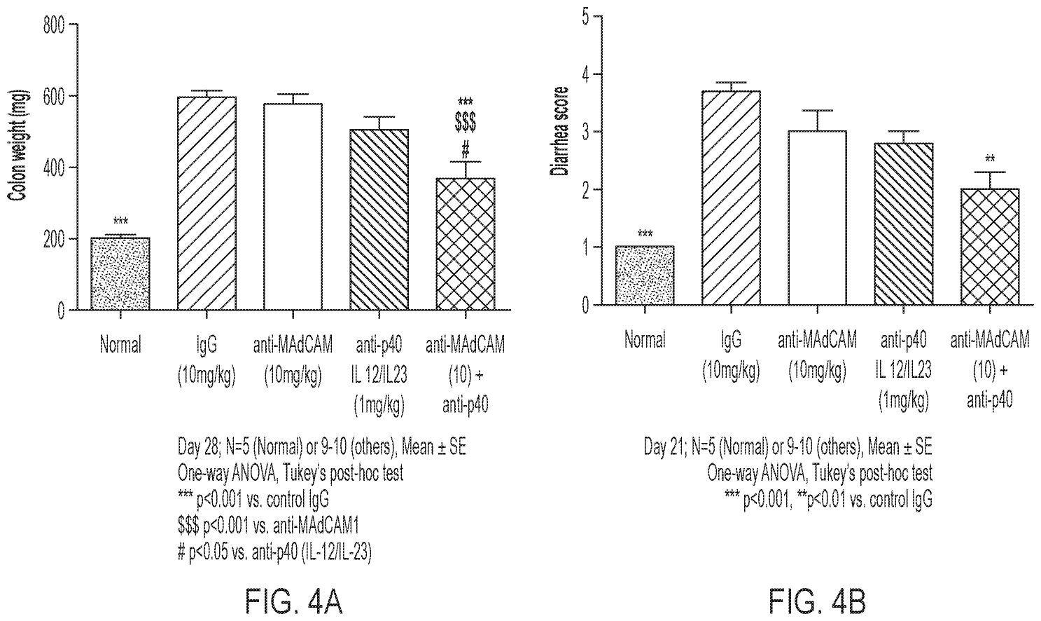

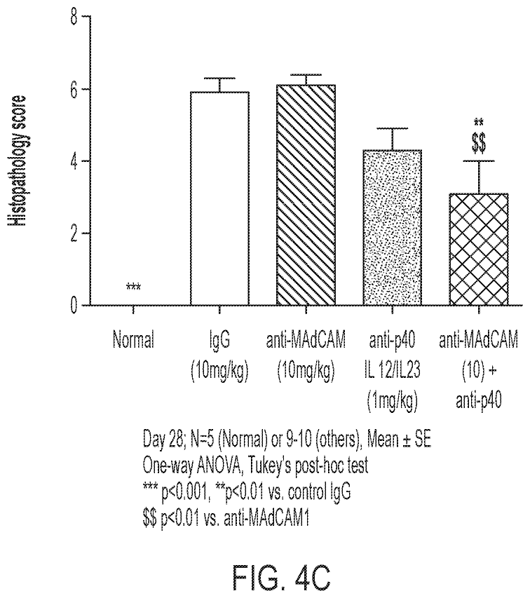

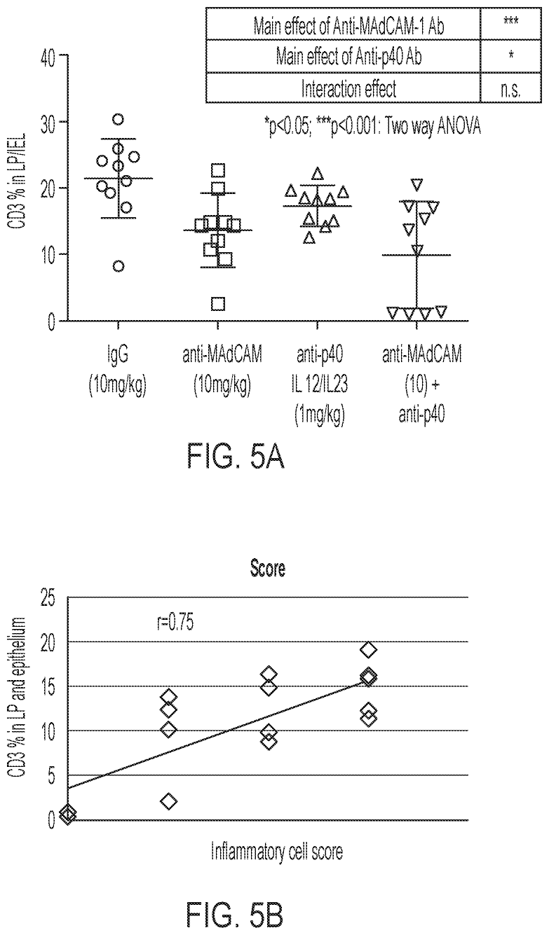

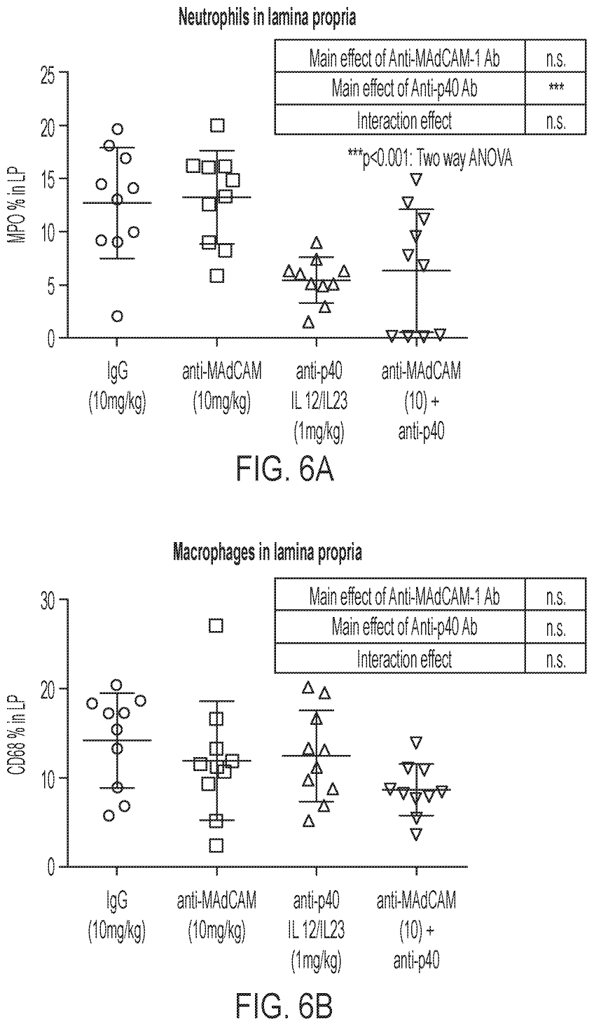

Results from the study are provided in FIGS. 4A, 4B, 4C, FIGS. 5A and 5B and in FIGS. 6A and 6B.

The combination of anti-MAdCAM antibody and an anti-p40 IL12/IL23 antibody showed superior effect on colon weight as shown in the results provided in FIG. 4A at day 28. Benefits of the combination therapy was also observed in the diarrhea score as shown in FIG. 4B at day 21, although the diarrhea score showed a positive trend at day 28 (data not shown) vs. the day 21 data shown in FIG. 4B. FIG. 4C provides a graph of a histopathology score at day 28 of H & E staining and showed a superior therapeutic effect of the combination therapy versus the antibody treatments alone or the controls.

Further, CD3 (T cell) staining in lamina propria/epithelium at Day 28 suggested that the predominant effect of anti-MAdCAM1 antibody was on T cell infiltration. The T cells in the lamina propria/epithelium were quantified for each treatment group, as described in FIG. 5A. T cell infiltration correlated with the total inflammatory cell score as described in FIG. 5B.

Myeloperoxidase (MPO) staining of neutrophils in lamina propria at day 28 indicated that anti-p40 antibody reduced MPO(+) neutrophils percentages in colon mucosa, and that the combination of an anti-MAdCAM1 inhibitor and an anti-p40 IL-23/IL-12 inhibitor showed a trend to decrease macrophages (CD68). These results are provided in FIGS. 6A and 6B.

The results from this study suggest that inhibiting the MAdCAM-1 pathway and inhibiting the IL-23 pathway results in complementary effects on adaptive and innate immune cells in colon mucosa. More specifically, anti-MAdCAM-1 and anti-p40 reduced CD3(+) T cell percentages in colon mucosa and a superior effect was observed with the combination. Anti-p40 antibody activity reduced MPO(+) neutrophils percentages in colon mucosa, while CD68(+) macrophages were not significantly affected by anti-MAdCAM-1, anti-p40, or their combination, (although a trend was observed with the latter).

### Example 2. Anti-MAdCAM-1 and Anti-p40 Combination Effect on mRNA Expression in TCT-Induced Mouse Chronic Colitis Model

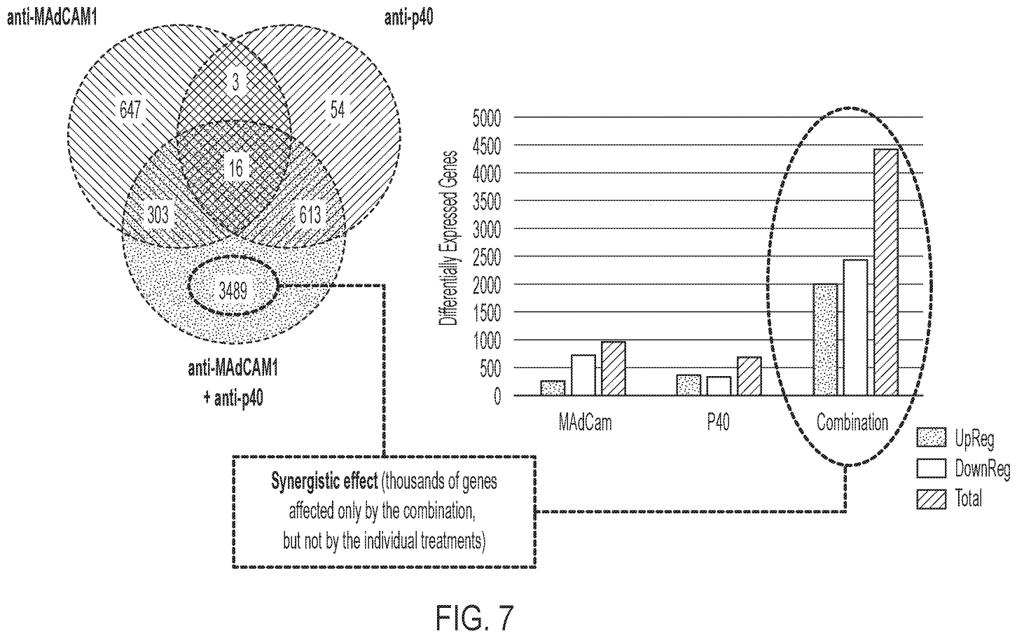

Gene expression in the colon was studied to determine the impact of anti-MADCAM-1, anti-p40 IL-23, and the combination of MAdCAM-1 and p40 IL-23 administration. A false discovery rate (FDR) of 0.05 was used to define differentially expressed genes (DEG). FIG. 7 provides a Venn diagram showing gene expression number. The results found that 4,422 genes had altered expression when the two therapies were combined, with about 3489 genes being expressed synergistically. The differential gene expression of treatment vs. controls is described in the graph of FIG. 7.

The combination of an anti-MAdCAM1 antibody and an anti-p40 antibody resulted in a number of genes showing a synergistic expression (either upregulated or downregulated), which were genes affected only by the combination of anti-MAdCAM-1 and anti-p40, but not by the individual treatments. These synergistically expressed genes included certain integrin chain mRNA expression, such as Itgal (αL chain/CD11a/LFA-1A), Itgb2 (β2 integrin chain/CD18), Itgax (αX chain/CD11c), Itga3, Itga9, and Itgb1bk. In particular, Itgal (αL chain/CD11a/LFA-1A), Itgb2 (β2 integrin chain/CD18), Itgax (αX chain/CD11c), Itga3, and Itgb1bk were synergistically downregulated. In contrast, Itga9 was shown to be synergistically upregulated.

In addition, certain cytokine genes were identified as having synergistic mRNA expression in the combination therapy, including Il21r, Il12rb1, Il12a, IL2ra, IL10ra, Il17re, Il34, Il18rap, Il1r11, Il1b, Il1r2, Il3ra, Il1f9, Il23a, Iltifb, Il6, Il18 bp, Il1a, Il15, and Il1r1. In particular, Il21r, Il12rb1, Il12a, IL2ra, IL10ra, Il34, Il18rap, Il1r11, Il1b, Il1r2, Il3ra, Il1f9, Il23a, Iltifb, Il6, Il18 bp, Il1a, and Il1r1 were synergistically downregulated. In contrast, Il17re and 1115 were synergistically upregulated. Other genes identified as having synergistic mRNA expression in the presence of the combination therapy (vs. the therapies alone) included Stat4, Stat2, and Cd3g (synergistically downregulated).

Overall, anti-MAdCAM-1 antibody treatment induced broad gene expression changes, including ITGB7, ITGAE, and interleukin receptors, when compared to control. Anti-p40 antibody treatment also induced broad gene expression changes that were distinct from those induced by anti-MAdCAM-1, when compared to control. Combining anti-MAdCAM-1 antibody treatment and anti-p40 antibody treatment induced broad gene expression of about 4422 genes, including increased downregulation in interleukins, integrins, stat, and Cd3. Synergistic inhibition of IL12/IL23A and complementary inhibition of ITGB7 and ITGA4 indicate that the combination therapy exerts an effect in the ‘right’ direction for both drug targets. That is, inhibiting one target does not increase the expression of the other.

### Example 3. Activity of Anti-MAdCAM-1 and Anti-p40 Antibody Combination in T-Cell Transfer (TCT) Induced Mouse Chronic Colitis Model

The experiment described in Example 1 was repeated using the treatment groups summarized in Table 1.

TABLE 1
Treatment Group Summary

| Normal | n = 5 |
|---|---|
| Isotype control antibody (10 + 1 mg/kg) | n = 10 |
| Anti-MAdCAM-1 antibody (10 mg/kg) | n = 10 |
| Anti-IL 12/23 p40 antibody (1 mg/kg) | n = 10 |
| Anti-MAdCAM-1 antibody (10 mg/kg) + | n = 10 |
| Anti-IL 12/23 p40 antibody (1 mg/kg) | |

Samples were collected and analyzed for α4β7+CD4+ T cell, diarrhea score, colon weight, histology of colon, and colon mRNA.

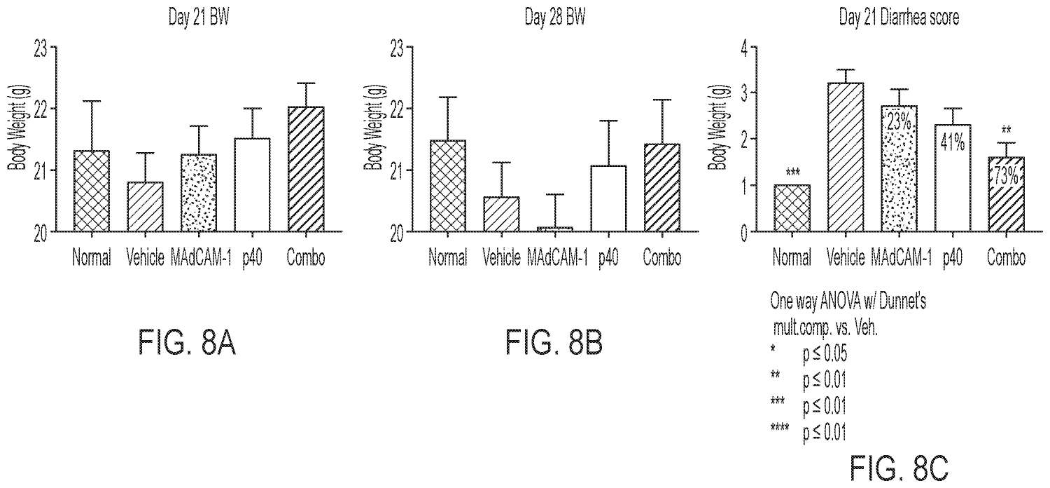

The combination demonstrated improved body weight at days 21, and 28, as shown in FIGS. 8A and 8B, respectively, and a significant superior effect on Day 21 diarrhea score (FIG. 8C) but not on day 28. Unlike the results described in Example 1, no superior effect was observed with the combination therapy on mouse colon weight or histopathology.

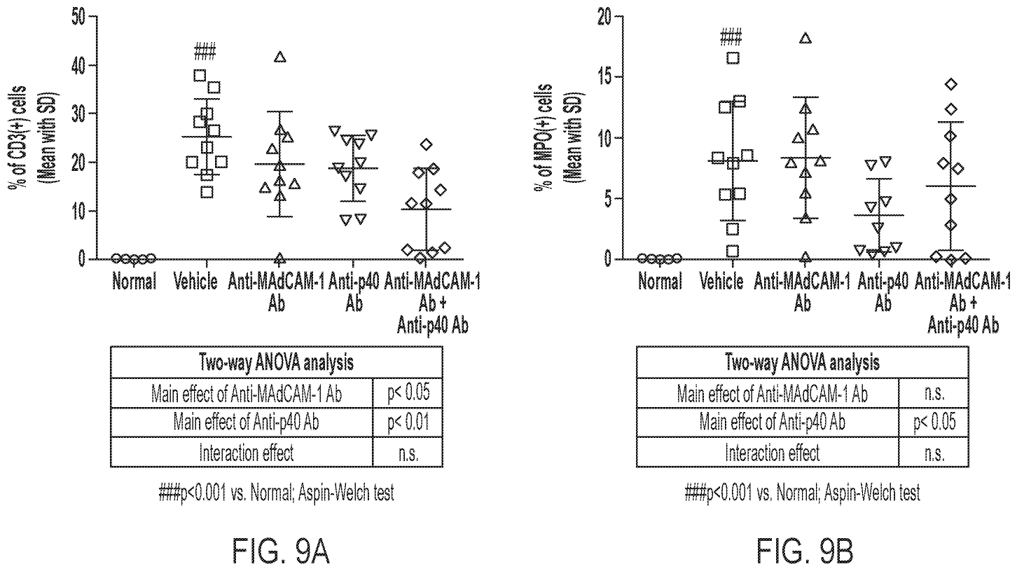

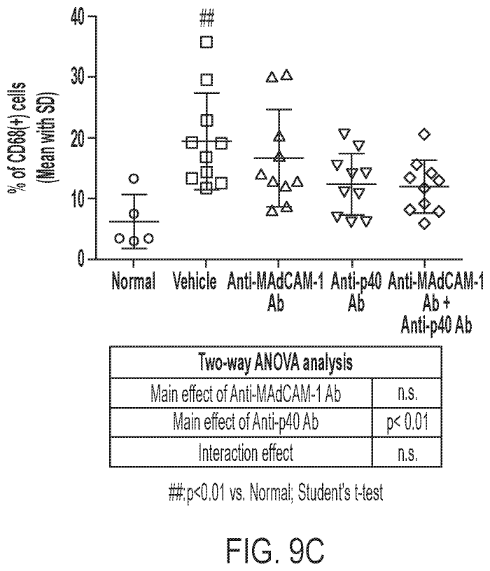

Effects of the combination therapy on T cell, neutrophil and macrophage infiltration was observed and was similar to the results provided in Example 1. The results from this repeat study are provided in FIG. 9A-9C. Anti-MAdCAM-1 antibody treatment and anti-p40 antibody treatment reduced CD3(+) T cell percentages in colon mucosa and superior effect was observed with the combination (as described in FIG. 9A). Further, anti-p40 antibody treatment reduced MPO(+) neutrophils in colon mucosa (as described in FIG. 9B), and CD68(+) macrophages were significantly affected by anti-p40 antibody treatment as well as the combination therapy (as described in FIG. 9C). Overall the results were very similar to those described from the study of Example 1.

In addition, gene expression in the colon was determined using RNAseq where an FDR, 0.05 indicated DEG. Approximately 343 genes were identified as having novel and synergistic gene expression in the combination therapy.

To summarize the studies described in Example 1 and above, significant superior effect on day 21 diarrhea score was observed in both studies. A trend toward improved day 21-28 body weight with the combo was shown in the repeat study (but not study 1), while a superior combo effect on colon weight and histopathology in first study did not repeat in the second study. Immunohistology results were very similar between studies—showing a complimentary effect on T cells, neutrophils, and macrophages in the colon. Overall, the two studies provided observations suggesting that inhibiting both the MAdCAM-1 pathway and the p40 subunit of IL-23 provides a significant activity. Further, RNAseq results indicated synergistic gene expression changes with the combination therapy.

### Example 4. Differential Modulation of the IL-22 Pathway in Vedolizumab Responder Versus Non-Responder IBD Patients

Vedolizumab (VDZ) is a monoclonal antibody (mAb) targeting α4β7 integrin approved for the treatment of moderate to severe Crohn's disease (CD) and ulcerative colitis (UC). Because of its unique safety profile, VDZ could be a good choice as the backbone of a combination biologic therapy to provide increased efficacy. Vedolizumab blocks the interaction between MAdCAM-1 and α4β7.

Anti-p19 (interleukin-23 (IL-23)) mAbs are efficacious in CD and UC, with an acceptable safety profile. However, it has not yet been shown whether a combination of anti-p19 plus VDZ will have additive efficacy.

Serum IL-22 levels have been shown to be higher in CD patients responding to anti-p19, suggesting that increased activity in the IL-22 pathway is associated with response to this therapy. Thus, it is hypothesized that if IL-22 is increased in VDZ non-responders it would support a combination with anti-p19 to provide superior efficacy.

Publicly available microarray data was originally generated in 41 moderate/severe bionaive UC patients (*). The analyses were done using the robust multichip average method, calculating log 2 expression values for IL22 and STAT5A probes in the array data, comparing VDZ responders, non-responders, and healthy controls. Serum IL-22 levels were assessed using Quantikine® ELISA (R&D Systems) in CD patients with moderate/severe disease from the Gemini-III trial; 59 remitters (VDZ-R) at weeks 6 and 10, and 59 non-remitters (VDZ-NR) at both time points (baseline average CDAI 297±49; median CRP 9.7 mg/L [3.7-24.5]; median FCP 583 mg/kg [229-1380]; 74.5% anti-TNF failures). Remitters and non-remitters were matched for baseline disease severity using CDAI and FCP.

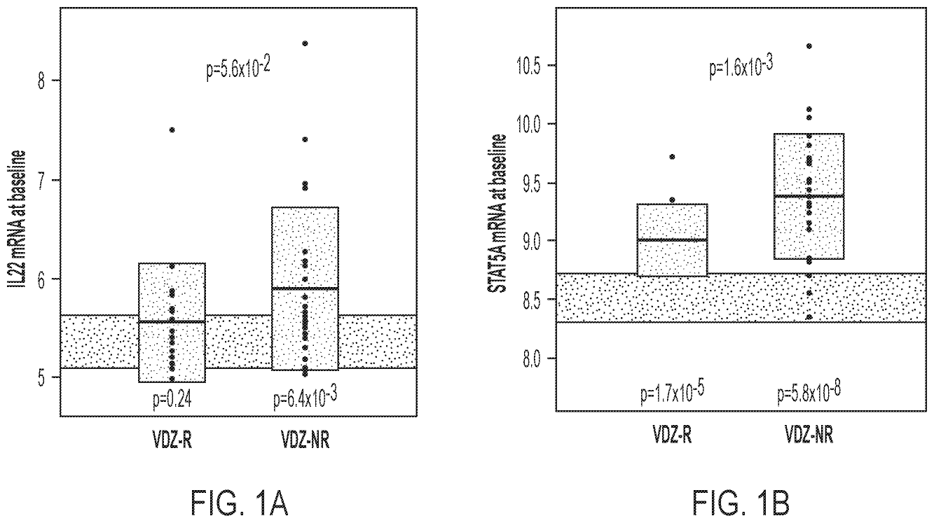

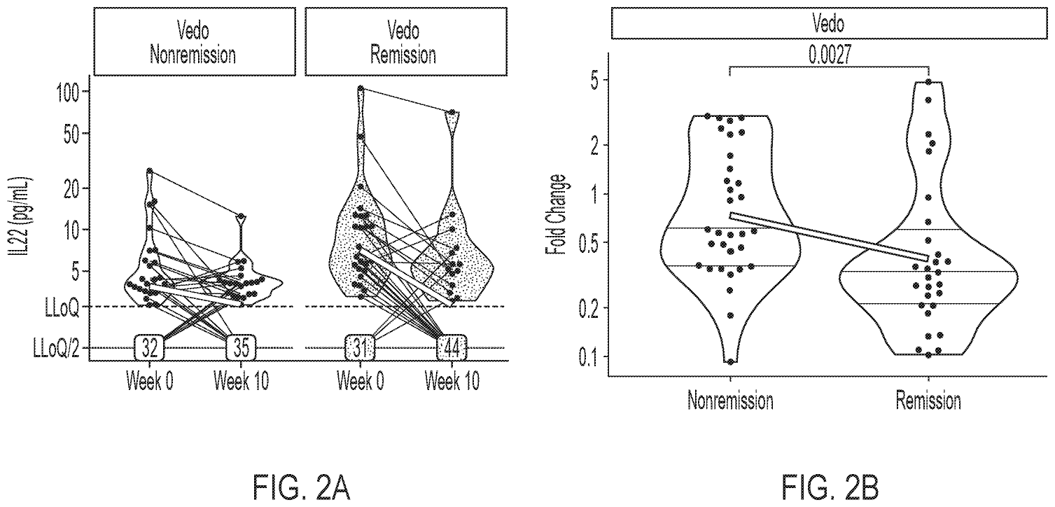

Baseline colon IL22 mRNA was significantly above healthy controls in VDZ non-responder UC patients (endoscopic subscore >1), while STAT5A mRNA (IL-22 signaling) was higher in VDZ non-responders compared to responders (FIG. 1). In addition, although baseline serum IL-22 was not different between VDZ remitters (CDAI<150) and non-remitters CD patients, decrease in IL-22 levels at week 10 was lower in VDZ non-remitters (FIG. 2).

Response to VDZ was preferentially seen in IBD patients without increased IL-22 pathway activity, suggesting that the addition of anti-p19/IL-23 therapy could be complementary to VDZ and potentially increase efficacy when administered as a combination regimen. The results suggest pilot clinical trials to test this hypothesis.

* (Arijs et al. Gut, 67: 43-52, 2018; Gene Expression Omnibus database accession number GSE73661)

### EQUIVALENTS

Those skilled in the art will recognize, or be able to ascertain using no more than routine experimentation, many equivalents to the specific embodiments of the invention described herein. Such equivalents are intended to be encompassed by the following claims. The contents of all references, patents and published patent applications cited throughout this application are incorporated herein by reference.

SEQUENCE TABLE

| SEQ ID | | |
|---|---|---|
| NO: | DESCRIPTION | SEQUENCE |
| 1 | Heavy chain (HC) | QVQLVQSGAEVKKPGASVKVSCKGSGYTFTSYWMHW |
| | variable region | VRQAPGQRLEWIGEIDPSESNTNYNQKFKGRVTLTVDIS |
| | (amino acid) | ASTAYMELSSLRSEDTAVYYCARGGYDGWDYAIDYW |
| | | GQGTLVTVSS |
| 2 | HC CDR1 (amino | SYWMH |
| | acid) | |
| 3 | HC CDR2 (amino | EIDPSESNTNYNQKFKG |
| | acid) | |
| 4 | HC CDR3 (amino | GGYDGWDYAIDY |
| | acid) | |
| 5 | Light chain (LC) | DVVMTQSPLSLPVTPGEPASISCRSSQSLAKSYGNTYLS |
| | variable region | WYLQKPGQSPQLLIYGISNRFSGVPDRFSGSGSGTDFTL |
| | (amino acid) | KISRVEAEDVGVYYCLQGTHQPYTFGQGTKVEIK |
| 6 | LC CDR1 (amino | RSSQSLAKSYGNTYLS |
| | acid) | |
| 7 | LC CDR2 (amino | GISNRFS |
| | acid) | |
| 8 | LC CDR3 (amino | LQGTHQPYT |
| | acid) | |
| 9 | Human IL-22 | ACAAGCAGAATCTTCAGAACAGGTTCTCCTTCCCCAGTCACC |
| | nucleic acid | AGTTGCTCGAGTTAGAATTGTCTGCAATGGCCGCCCTGCAGA |
| | sequence | AATCTGTGAGCTCTTTCCTTATGGGGACCCTGGCCACCAGCT |
| | GenBank | GCCTCCTTCTCTTGGCCCTCTTGGTACAGGGAGGAGCAGCTG |
| | Accession Number | CGCCCATCAGCTCCCACTGCAGGCTTGACAAGTCCAACTTCC |
| | NM_020525.5 | AGCAGCCCTATATCACCAACCGCACCTTCATGCTGGCTAAGG |
| | | AGGCTAGCTTGGCTGATAACAACACAGACGTTCGTCTCATTG |
| | | GGGAGAAACTGTTCCACGGAGTCAGTATGAGTGAGCGCTGCT |
| | | ATCTGATGAAGCAGGTGCTGAACTTCACCCTTGAAGAAGTGC |
| | | TGTTCCCTCAATCTGATAGGTTCCAGCCTTATATGCAGGAGG |
| | | TGGTGCCCTTCCTGGCCAGGCTCAGCAACAGGCTAAGCACAT |
| | | GTCATATTGAAGGTGATGACCTGCATATCCAGAGGAATGTGC |
| | | AAAAGCTGAAGGACACAGTGAAAAAGCTTGGAGAGAGTGGAG |
| | | AGATCAAAGCAATTGGAGAACTGGATTTGCTGTTTATGTCTC |
| | | TGAGAAATGCCTGCATTTGACCAGAGCAAAGCTGAAAAATGA |
| | | ATAACTAACCCCCTTTCCCTGCTAGAAATAACAATTAGATGC |
| | | CCCAAAGCGATTTTTTTTAACCAAAAGGAAGATGGGAAGCCA |
| | | AACTCCATCATGATGGGTGGATTCCAAATGAACCCCTGCGTT |
| | | AGTTACAAAGGAAACCAATGCCACTTTTGTTTATAAGACCAG |
| | | AAGGTAGACTTTCTAAGCATAGATATTTATTGATAACATTTC |
| | | ATTGTAACTGGTGTTCTATACACAGAAAACAATTTATTTTTT |
| | | AAATAATTGTCTTTTTCCATAAAAAAGATTACTTTCCATTCC |
| | | TTTAGGGGAAAAAACCCCTAAATAGCTTCATGTTTCCATAAT |
| | | CAGTACTTTATATTTATAAATGTATTTATTATTATTATAAGA |
| | | CTGCATTTTATTTATATCATTTTATTAATATGGATTTATTTA |
| | | TAGAAACATCATTCGATATTGCTACTTGAGTGTAAGGCTAAT |
| | | ATTGATATTTATGACAATAATTATAGAGCTATAACATGTTTA |
| | | TTTGACCTCAATAAACACTTGGATATCCTAA |
| 10 | Human IL-22 | MAALQKSVSSFLMGTLATSCLLLLALLVQGGAAAPISSHCRL |
| | protein (precursor) | DKSNFQQPYITNRTFMLAKEASLADNNTDVRLIGEKLFHGVS |
| | amino acid | MSERCYLMKQVLNFTLEEVLFPQSDRFQPYMQEWPFLARLS |
| | sequence | NRLSTCHIEGDDLHIQRNVQKLKDTVKKLGESGEIKAIGELD |
| | GenPept Accession | LLFMSLRNACI |
| | number | |
| | NP_065386.1 | |
| 11 | Human STAT5A | AGACAGGATATTCACTGCTGTGGCAAGGCCTGTAGAGAGTTT |
| | nucleic acid | CGAAGTTAGGAGGACTCAAGACGGTCCCTCCCTGGACTTTTC |
| | sequence (variant | TGAAGGGGCTCAAAAGATGACACGCGCCAGAGCTGGAAGGCG |
| | 2) | TCGCCAATTGGTCCACTTTTCCCTCCTCCCTTTTTGCGGATG |
| | GenBank | AGAAAACTGAGGCCCAGGTTTGGGATTTCCAGAGCCCGGGAT |
| | Accession number | TTCCCGGCAACGCCCGACAACCACATTCCCCCGGCTATTCTG |
| | NM_003152.3 | ACCCGCCCCGGTTCCGGGACGCTCCCTGGGAGCCGCCGCCGA |
| | | GGGCCTGCTGGGACTCCCGGGGGACCCCGCCGTCGGGGCAGC |
| | | CCCCACGCCCGGCGCCGCCCGCCGGGAACGGCCGCCGCTGTT |
| | | GCGCACTTGCAGGGGAGCCGGCGACTGAGGGCGAGGCAGGGA |
| | | GGGAGCAAGCGGGGCTGGGAGGGCTGCTGGCGCGGGCTCGCG |
| | | CGCTGTGTATGGTCTATCGCAGGCAGCTGACCTTTGAGGAGG |
| | | AAATCGCTGCTCTCCGCTCCTTCCTGTAGTAACAGCCGCCGC |
| | | TGCCGCCGCCGCCAGGAACCCCGGCCGGGAGCGAGAGCCGCG |
| | | GGGCGCAGAGCCGGCCCGGCTGCCGGACGGTGCGGCCCCACC |
| | | AGGTGAACGGCCATGGCGGGCTGGATCCAGGCCCAGCAGCTG |
| | | CAGGGAGACGCGCTGCGCCAGATGCAGGTGCTGTACGGCCAG |
| | | CACTTCCCCATCGAGGTCCGGCACTACTTGGCCCAGTGGATT |
| | | GAGAGCCAGCCATGGGATGCCATTGACTTGGACAATCCCCAG |
| | | GACAGAGCCCAAGCCACCCAGCTCCTGGAGGGCCTGGTGCAG |
| | | GAGCTGCAGAAGAAGGCGGAGCACCAGGTGGGGGAAGATGGG |
| | | TTTTTACTGAAGATCAAGCTGGGGCACTACGCCACGCAGCTC |
| | | CAGAAAACATATGACCGCTGCCCCCTGGAGCTGGTCCGCTGC |
| | | ATCCGGCACATTCTGTACAATGAACAGAGGCTGGTCCGAGAA |
| | | GCCAACAATTGCAGCTCTCCGGCTGGGATCCTGGTTGACGCC |
| | | ATGTCCCAGAAGCACCTTCAGATCAACCAGACATTTGAGGAG |
| | | CTGCGACTGGTCACGCAGGACACAGAGAATGAGCTGAAGAAA |
| | | CTGCAGCAGACTCAGGAGTACTTCATCATCCAGTACCAGGAG |
| | | AGCCTGAGGATCCAAGCTCAGTTTGCCCAGCTGGCCCAGCTG |
| | | AGCCCCCAGGAGCGTCTGAGCCGGGAGACGGCCCTCCAGCAG |
| | | AAGCAGGTGTCTCTGGAGGCCTGGTTGCAGCGTGAGGCACAG |
| | | ACACTGCAGCAGTACCGCGTGGAGCTGGCCGAGAAGCACCAG |
| | | AAGACCCTGCAGCTGCTGCGGAAGCAGCAGACCATCATCCTG |
| | | GATGACGAGCTGATCCAGTGGAAGCGGCGGCAGCAGCTGGCC |
| | | GGGAACGGCGGGCCCCCCGAGGGCAGCCTGGACGTGCTACAG |
| | | TCCTGGTGTGAGAAGTTGGCCGAGATCATCTGGCAGAACCGG |
| | | CAGCAGATCCGCAGGGCTGAGCACCTCTGCCAGCAGCTGCCC |
| | | ATCCCCGGCCCAGTGGAGGAGATGCTGGCCGAGGTCAACGCC |
| | | ACCATCACGGACATTATCTCAGCCCTGGTGACCAGCACATTC |
| | | ATCATTGAGAAGCAGCCTCCTCAGGTCCTGAAGACCCAGACC |
| | | AAGTTTGCAGCCACCGTACGCCTGCTGGTGGGCGGGAAGCTG |
| | | AACGTGCACATGAATCCCCCCCAGGTGAAGGCCACCATCATC |
| | | AGTGAGCAGCAGGCCAAGTCTCTGCTTAAAAATGAGAACACC |
| | | CGCAACGAGTGCAGTGGTGAGATCCTGAACAACTGCTGCGTG |
| | | ATGGAGTACCACCAAGCCACGGGCACCCTCAGTGCCCACTTC |
| | | AGGAACATGTCACTGAAGAGGATCAAGCGTGCTGACCGGCGG |
| | | GGTGCAGAGTCCGTGACAGAGGAGAAGTTCACAGTCCTGTTT |
| | | GAGTCTCAGTTCAGTGTTGGCAGCAATGAGCTTGTGTTCCAG |
| | | GTGAAGACTCTGTCCCTACCTGTGGTTGTCATCGTCCACGGC |
| | | AGCCAGGACCACAATGCCACGGCTACTGTGCTGTGGGACAAT |
| | | GCCTTTGCTGAGCCGGGCAGGGTGCCATTTGCCGTGCCTGAC |
| | | AAAGTGCTGTGGCCGCAGCTGTGTGAGGCGCTCAACATGAAA |
| | | TTCAAGGCCGAAGTGCAGAGCAACCGGGGCCTGACCAAGGAG |
| | | AACCTCGTGTTCCTGGCGCAGAAACTGTTCAACAACAGCAGC |
| | | AGCCACCTGGAGGACTACAGTGGCCTGTCCGTGTCCTGGTCC |
| | | CAGTTCAACAGGGAGAACTTGCCGGGCTGGAACTACACCTTC |
| | | TGGCAGTGGTTTGACGGGGTGATGGAGGTGTTGAAGAAGCAC |
| | | CACAAGCCCCACTGGAATGATGGGGCCATCCTAGGTTTTGTG |
| | | AATAAGCAACAGGCCCACGACCTGCTCATCAACAAGCCCGAC |
| | | GGGACCTTCTTGTTGCGCTTTAGTGACTCAGAAATCGGGGGC |
| | | ATCACCATCGCCTGGAAGTTTGACTCCCCGGAACGCAACCTG |
| | | TGGAACCTGAAACCATTCACCACGCGGGATTTCTCCATCAGG |
| | | TCCCTGGCTGACCGGCTGGGGGACCTGAGCTATCTCATCTAT |
| | | GTGTTTCCTGACCGCCCCAAGGATGAGGTCTTCTCCAAGTAC |
| | | TACACTCCTGTGCTGGCTAAAGCTGTTGATGGATATGTGAAA |
| | | CCACAGATCAAGCAAGTGGTCCCTGAGTTTGTGAATGCATCT |
| | | GCAGATGCTGGGGGCAGCAGCGCCACGTACATGGACCAGGCC |
| | | CCCTCCCCAGCTGTGTGCCCCCAGGCTCCCTATAACATGTAC |
| | | CCACAGAACCCTGACCATGTACTCGATCAGGATGGAGAATTC |
| | | GACCTGGATGAGACCATGGATGTGGCCAGGCACGTGGAGGAA |
| | | CTCTTACGCCGACCAATGGACAGTCTTGACTCCCGCCTCTCG |
| | | CCCCCTGCCGGTCTTTTCACCTCTGCCAGAGGCTCCCTCTCA |
| | | TGAATGTTTGAATCCCACGCTTCTCTTTGGAAACAATATGCA |
| | | ATGTGAAGCGGTCGTGTTGTGAGTTTAGTAAGGCTGTGTACA |
| | | CTGACACCTTTGCAGGCATGCATGTGCTTGTGTGTGTGTGTG |
| | | TGTGTGTGTCCTTGTGCATGAGCTACGCCTGCCTCCCCTGTG |
| | | CAGTCCTGGGATGTGGCTGCAGCAGCGGTGGCCTCTTTTCAG |
| | | ATCATGGCATCCAAGAGTGCGCCGAGTCTGTCTCTGTCATGG |
| | | TAGAGACCGAGCCTCTGTCACTGCAGGCACTCAATGCAGCCA |
| | | GACCTATTCCTCCTGGGCCCCTCATCTGCTCAGCAGCTATTT |
| | | GAATGAGATGATTCAGAAGGGGAGGGGAGACAGGTAACGTCT |
| | | GTAAGCTGAAGTTTCACTCCGGAGTGAGAAGCTTTGCCCTCC |
| | | TAAGAGAGAGAGACAGAGAGACAGAGAGAGAGAAAGAGAGAG |
| | | TGTGTGGGTCTATGTAAATGCATCTGTCCTCATGTGTTGATG |
| | | TAACCGATTCATCTCTCAGAAGGGAGGCTGGGGTTCATTTTC |
| | | GAGTAGTATTTTATACTTTAGTGAACGTGGACTCCAGACTCT |
| | | CTGTGAACCCTATGAGAGCGCGTCTGGGCCCGGCCATGTCCT |
| | | TAGCACAGGGGGGCCGCCGGTTTGAGTGAGGGTTTCTGAGCT |
| | | GCTCTGAATTAGTCCTTGCTTGGCTGCTTGGCCTTGGGCTTC |
| | | ATTCAAGTCTATGATGCTGTTGCCCACGTTTCCCGGGATATA |
| | | TATTCTCTCCCCTCCGTTGGGCCCCAGCCTTCTTTGCTTGCC |
| | | TCTCTGTTTGTAACCTTGTCGACAAAGAGGTAGAAAAGATTG |
| | | GGTCTAGGATATGGTGGGTGGACAGGGGCCCCGGGACTTGGA |
| | | GGGTTGGTCCTCTTGCCTCCTGGAAAAAACAAAAACAAAAAA |
| | | CTGCAGTGAAAGACAAGCTGCAAATCAGCCATGTGCTGCGTG |
| | | CCTGTGGAATCTGGAGTGAGGGGTAAAAGCTGATCTGGTTTG |
| | | ACTCCGCTGGAGGTGGGGCCTGGAGCAGGCCTTGCGCTGTTG |
| | | CGTAACTGGCTGTGTTCTGGTGAGGCCTTGCTCCCAACCCCA |
| | | CACGCTCCTCCCTCTGAGGCTGTAGGACTCGCAGTCAGGGGC |
| | | AGCTGACCATGGAAGATTGAGAGCCCAAGGTTTAAACTTCTC |
| | | TGAAGGGAGGTGGGGATGAGAAGAGGGGTTTTTTTGTACTTT |
| | | GTACAAAGACCACACATTTGTGTAAACAGTGTTTTGGAATAA |
| | | AATATTTTTTTCATAAAAAAAAAAAAAAAA |
| 12 | Human STAT5A | MAGWIQAQQLQGDALRQMQVLYGQHFPIEVRHYLAQWIESQP |
| | amino acid | WDAIDLDNPQDRAQATQLLEGLVQELQKKAEHQVGEDGFLLK |
| | sequence (isoform | IKLGHYATQLQKTYDRCPLELVRCIRHILYNEQRLVREANNC |
| | 1) | SSPAGILVDAMSQKHLQINQTFEELRLVTQDTENELKKLQQT |
| | GenPept Accession | QEYFIIQYQESLRIQAQFAQLAQLSPQERLSRETALQQKQVS |
| | number | LEAWLQREAQTLQQYRVELAEKHQKTLQLLRKQQTIILDDEL |
| | NP_003143.2 | IQWKRRQQLAGNGGPPEGSLDVLQSWCEKLAEIIWQNRQQIR |
| | | RAEHLCQQLPIPGPVEEMLAEVNATITDIISALVTSTFIIEK |
| | | QPPQVLKTQTKFAATVRLLVGGKLNVHMNPPQVKAT11SEQQ |
| | | AKSLLKNENTRNECSGEILNNCCVMEYHQATGTLSAHFRNMS |
| | | LKRIKRADRRGAESVTEEKFTVLFESQFSVGSNELVFQVKTL |
| | | SLPVVVIVHGSQDHNATATVLWDNAFAEPGRVPFAVPDKVLW |
| | | PQLCEALNMKFKAEVQSNRGLTKENLVFLAQKLFNNSSSHLE |
| | | DYSGLSVSWSQFNRENLPGWNYTFWQWFDGVMEVLKKHHKPH |
| | | WNDGAILGFVNKQQAHDLLINKPDGTFLLRFSDSEIGGITIA |
| | | WKFDSPERNLWNLKPFTTRDFSIRSLADRLGDLSYLIYVFPD |
| | | RPKDEVFSKYYTPVLAKAVDGYVKPQIKQWPEFVNASADAG |
| | | GSSATYMDQAPSPAVCPQAPYNMYPQNPDHVLDQDGEFDLDE |
| | | TMDVARHVEELLRRPMDSLDSRLSPPAGLFTSARGSLS |
| 13 | Human IL-23B | mchqqlviswfslvflasplvaiwelkkdvyvveldwypdap |
| | (p40) subunit | gemvvltcdtpeedgitwtldqssevlgsgktltiqvkefgd |
| | amino acid | agqytchkggevlshsllllhkkedgiwstdilkdqkepknk |
| | sequence | tflrceaknysgrftcwwlttistdltfsvkssrgssdpqgv |
| | RefSeq. Accession | tcgaatlsaervrgdnkeyeysvecqedsacpaaeeslpiev |
| | No. NP_002178.2 | mvdavhklkyenytssffirdiikpdppknlqlkplknsrqv |
| | | evsweypdtwstphsyfsitfcvqvqgkskrekkdrvftdkt |
| | | satvicrknasisvraqdryyssswsewasvpcs |
| 14 | Human IL-23A | mlgsravmlllllpwtaqgravpggsspawtqcqqlsqklct |
| | (p19) subunit | lawsahplvghmdlreegdeettndvphiqcgdgcdpqglrd |
| | amino acid | nsqfclqrihqglifyekllgsdiftgepsllpdspvgqlha |
| | sequence | sllglsqllqpeghhwetqqipslspsqpwqrlllrfkilrs |
| | RefSeq. Accession | lqafvavaarvfahgaatlsp |
| | No. NP_057668.1 | |

1.-4. (canceled)

5. A method of treating a human patient in need thereof, said method comprising administering a humanized anti-α4β7 antibody and an IL-23 inhibitor to the human patient,
wherein the humanized anti-α4β7 antibody is an IgG1 antibody; comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

6. The method of claim 5, wherein the human patient has an autoimmune disease.

7. The method of claim 6, wherein the autoimmune disease is arthritis or psoriasis.

8. The method of claim 6, wherein the autoimmune disease is rheumatoid arthritis, juvenile arthritis, psoriatic arthritis, or axial spondyloarthritis.

9. The method of claim 5, wherein the human patient has inflammatory bowel disease (IBD).

10. (canceled)

11. The method of claim 9, wherein the IBD is ulcerative colitis or Crohn's disease.

12.-13. (canceled)

14. The method of claim 5, wherein the humanized anti-α4β7 antibody is administered before the IL-23 inhibitor, after the IL-23 inhibitor, or concomitantly with the IL-23 inhibitor.

15.-16. (canceled)

17. The method of claim 1, wherein the humanized anti-α4β7 antibody comprises a heavy chain variable domain comprising an amino acid sequence as set forth in SEQ ID NO: 1, and comprises a light chain variable domain comprising an amino acid sequence as set forth in SEQ ID NO: 5 or wherein the humanized anti-α4β7 antibody is vedolizumab.

18.-19. (canceled)

20. The method of claim 5 of claim, wherein the human patient is administered a first dose of 300 mg of the humanized anti-α4β7 antibody at week 0, followed by a second dose of 300 mg of the humanized anti-α4β7 antibody at week 2, followed by third dose of 300 mg of the humanized anti-α4β7 antibody at week 6.

21.-25. (canceled)

26. The method of claim 20, further comprising administering 108 mg of the humanized anti-α4β7 antibody to the human patient every two weeks beginning eight weeks after the third dose.

27. The method of claim 5 of claim, wherein the human patient is administered a first dose of 300 mg of the humanized anti-α4β7 antibody at week 0, followed by a second dose of 300 mg of the humanized anti-α4β7 antibody at week 2, followed by third dose of 108 mg of the humanized anti-α4β7 antibody at week 6, followed by a 108 mg dose every two weeks thereafter.

28.-29. (canceled)

30. The method of claim 5, wherein the IL-23 inhibitor is selected from the group consisting of an antibody that binds to the p19 subunit of IL-23, an antibody that binds to the p40 subunit of IL-23, and an antibody that binds to IL-23R.

31. (canceled)

32. The method of claim 5, wherein the IL-23 inhibitor is risankizumab, ustekinumab, guselkumab, or tildrakizumab.

33. (canceled)

34. The method of claim 5, wherein the human patient has been characterized as a nonresponder or nonremitter at week 6 and/or week 10 after beginning treatment with the humanized anti-α4β7 antibody, or as having an elevated level of serum IL-22 at baseline or week 6 after beginning treatment with the humanized anti-α4β7 antibody.

35.-37. (canceled)

38. The method of claim 5, wherein serum IL-22 level of the human patient decreases none, less than two-fold, or less than three-fold from baseline to week 10 after beginning treatment with the anti-α4β7 antibody, baseline to week 6 after beginning treatment with the anti-α4β7 antibody, or week 6 to week 10 after beginning treatment with the anti-α4β7 antibody; or
wherein the patient has been characterized as having an elevated level of serum IL-22 at baseline or week 6 after beginning treatment with the anti-α4β7 antibody, and wherein serum IL-22 level decreases none, less than two-fold, or less than three-fold from baseline to week 10, baseline to week 6 or week 6 to week 10.

39. (canceled)

40. The method of claim 5, wherein the patient is characterized as having elevated levels of IL-22 and/or STAT5A as compared to a control level.

41. The method of claim 40, wherein
the control level is a level from one or more of a subject that does not suffer from IBD, a healthy subject, a non-inflamed colonic tissue, or a non-colonic tissue from the patient, and/or
the patient's IL-22 and/or STAT5A level is measured before treatment or on the first day of treatment with the anti-α4β7 antibody.

42.-43. (canceled)

44. The method of claim 34, wherein the patient's IL-22 and/or STAT5A nucleic acid and/or protein level is measured.

45. The method of claim 40, wherein the IL-22 and/or STAT5A level is elevated 5%, 10%, 15%, 20%, 25%, 30%, 40%, 50%, 60%, 70%, 80%, 90%, 100%, or more as compared to a control level.

46. A method of treating an inflammatory bowel disease in a patient in need thereof, comprising administering to the patient an anti-α4β7 antibody and an antibody that binds to the p19 subunit of IL-23 or to the p40 subunit of IL-23, wherein the anti-α4β7 antibody is administered a first dose of 300 mg of the anti-α4β7 antibody at week 0, followed by a second dose of 300 mg of the anti-α4β7 antibody at week 2, a third dose of 300 mg of the anti-α4β7 antibody at week 6, followed by a 300 mg dose of the anti-α4β7 antibody to the human patient every eight weeks beginning 8 weeks after the third dose,
wherein the anti-α4β7 antibody comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6, and
wherein the patient is characterized as having elevated levels of IL-22 and/or STAT5A compared to a control level.

47. (canceled)

48. A method of treating an inflammatory bowel disease in a patient in need thereof, comprising administering to the patient an anti-α4β7 antibody and an antibody that binds to the p19 subunit of IL-23 or to the p40 subunit of IL-23, wherein the anti-α4β7 antibody is administered a first dose of 300 mg of the anti-α4β7 antibody at week 0, followed by a second dose of 300 mg of the anti-α4β7 antibody at week 2, followed by third dose of 108 mg of the anti-α4β7 antibody at week 6, followed by a 108 mg dose every two weeks thereafter,
wherein the anti-α4β7 antibody comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6, and
wherein the patient is characterized as having elevated levels of IL-22 and/or STAT5A as compared to a control level.

49. (canceled)

50. The method of claim 46, wherein the control level is a level from one or more of a subject that does not suffer from IBD, a healthy subject, a non-inflamed colonic tissue, or a non-colonic tissue from the patient.

51. The method of claim 48, wherein the control level is a level from one or more of a subject that does not suffer from IBD, a healthy subject, a non-inflamed colonic tissue, or a non-colonic tissue from the patient.


SEQUENCE LISTING (14 sequences)

SEQ ID NO: 1 - protein, 121 aa, Artificial Sequence [sequence omitted]

SEQ ID NO: 2 - protein, 5 aa, Artificial Sequence
  Description of Artificial Sequence Synthetic peptide
SYWMH

SEQ ID NO: 3 - protein, 17 aa, Artificial Sequence
  Description of Artificial Sequence Synthetic peptide
EIDPSESNTNYNQKFKG

SEQ ID NO: 4 - protein, 12 aa, Artificial Sequence
  Description of Artificial Sequence Synthetic peptide
GGYDGWDYAIDY

SEQ ID NO: 5 - protein, 112 aa, Artificial Sequence [sequence omitted]

SEQ ID NO: 6 - protein, 16 aa, Artificial Sequence
  Description of Artificial Sequence Synthetic peptide
RSSQSLAKSYGNTYLS

SEQ ID NO: 7 - protein, 7 aa, Artificial Sequence
  Description of Artificial Sequence Synthetic peptide
GISNRFS

SEQ ID NO: 8 - protein, 9 aa, Artificial Sequence
  Description of Artificial Sequence Synthetic peptide
LQGTHQPYT

SEQ ID NO: 9 - DNA, 1165 bp, Homo sapiens
acaagcagaatcttcagaacaggttctccttccccagtcaccagttgctcgagttagaattgtctgcaatggccgccctgcagaaatctgtgagctctttccttatggggaccctggccaccagctgcctccttctcttggccctcttggtacagggaggagcagctgcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcatttgaccagagcaaagctgaaaaatgaataactaaccccctttccctgctagaaataacaattagatgccccaaagcgattttttttaaccaaaaggaagatgggaagccaaactccatcatgatgggtggattccaaatgaacccctgcgttagttacaaaggaaaccaatgccacttttgtttataagaccagaaggtagactttctaagcatagatatttattgataacatttcattgtaactggtgttctatacacagaaaacaatttattttttaaataattgtctttttccataaaaaagattactttccattcctttaggggaaaaaacccctaaatagcttcatgtttccataatcagtactttatatttataaatgtatttattattattataagactgcattttatttatatcattttattaatatggatttatttatagaaacatcattcgatattgctacttgagtgtaaggctaatattgatatttatgacaataattatagagctataacatgtttatttgacctcaataaacacttggatatcctaa

SEQ ID NO: 10 - protein, 179 aa, Homo sapiens
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 11 - DNA, 4314 bp, Homo sapiens
agacaggatattcactgctgtggcaaggcctgtagagagtttcgaagttaggaggactcaagacggtccctccctggacttttctgaaggggctcaaaagatgacacgcgccagagctggaaggcgtcgccaattggtccacttttccctcctccctttttgcggatgagaaaactgaggcccaggtttgggatttccagagcccgggatttcccggcaacgcccgacaaccacattcccccggctattctgacccgccccggttccgggacgctccctgggagccgccgccgagggcctgctgggactcccgggggaccccgccgtcggggcagcccccacgcccggcgccgcccgccgggaacggccgccgctgttgcgcacttgcaggggagccggcgactgagggcgaggcagggagggagcaagcggggctgggagggctgctggcgcgggctcgcgcgctgtgtatggtctatcgcaggcagctgacctttgaggaggaaatcgctgctctccgctccttcctgtagtaacagccgccgctgccgccgccgccaggaaccccggccgggagcgagagccgcggggcgcagagccggcccggctgccggacggtgcggccccaccaggtgaacggccatggcgggctggatccaggcccagcagctgcagggagacgcgctgcgccagatgcaggtgctgtacggccagcacttccccatcgaggtccggcactacttggcccagtggattgagagccagccatgggatgccattgacttggacaatccccaggacagagcccaagccacccagctcctggagggcctggtgcaggagctgcagaagaaggcggagcaccaggtgggggaagatgggtttttactgaagatcaagctggggcactacgccacgcagctccagaaaacatatgaccgctgccccctggagctggtccgctgcatccggcacattctgtacaatgaacagaggctggtccgagaagccaacaattgcagctctccggctgggatcctggttgacgccatgtcccagaagcaccttcagatcaaccagacatttgaggagctgcgactggtcacgcaggacacagagaatgagctgaagaaactgcagcagactcaggagtacttcatcatccagtaccaggagagcctgaggatccaagctcagtttgcccagctggcccagctgagcccccaggagcgtctgagccgggagacggccctccagcagaagcaggtgtctctggaggcctggttgcagcgtgaggcacagacactgcagcagtaccgcgtggagctggccgagaagcaccagaagaccctgcagctgctgcggaagcagcagaccatcatcctggatgacgagctgatccagtggaagcggcggcagcagctggccgggaacggcgggccccccgagggcagcctggacgtgctacagtcctggtgtgagaagttggccgagatcatctggcagaaccggcagcagatccgcagggctgagcacctctgccagcagctgcccatccccggcccagtggaggagatgctggccgaggtcaacgccaccatcacggacattatctcagccctggtgaccagcacattcatcattgagaagcagcctcctcaggtcctgaagacccagaccaagtttgcagccaccgtacgcctgctggtgggcgggaagctgaacgtgcacatgaatcccccccaggtgaaggccaccatcatcagtgagcagcaggccaagtctctgcttaaaaatgagaacacccgcaacgagtgcagtggtgagatcctgaacaactgctgcgtgatggagtaccaccaagccacgggcaccctcagtgcccacttcaggaacatgtcactgaagaggatcaagcgtgctgaccggcggggtgcagagtccgtgacagaggagaagttcacagtcctgtttgagtctcagttcagtgttggcagcaatgagcttgtgttccaggtgaagactctgtccctacctgtggttgtcatcgtccacggcagccaggaccacaatgccacggctactgtgctgtgggacaatgcctttgctgagccgggcagggtgccatttgccgtgcctgacaaagtgctgtggccgcagctgtgtgaggcgctcaacatgaaattcaaggccgaagtgcagagcaaccggggcctgaccaaggagaacctcgtgttcctggcgcagaaactgttcaacaacagcagcagccacctggaggactacagtggcctgtccgtgtcctggtcccagttcaacagggagaacttgccgggctggaactacaccttctggcagtggtttgacggggtgatggaggtgttgaagaagcaccacaagccccactggaatgatggggccatcctaggttttgtgaataagcaacaggcccacgacctgctcatcaacaagcccgacgggaccttcttgttgcgctttagtgactcagaaatcgggggcatcaccatcgcctggaagtttgactccccggaacgcaacctgtggaacctgaaaccattcaccacgcgggatttctccatcaggtccctggctgaccggctgggggacctgagctatctcatctatgtgtttcctgaccgccccaaggatgaggtcttctccaagtactacactcctgtgctggctaaagctgttgatggatatgtgaaaccacagatcaagcaagtggtccctgagtttgtgaatgcatctgcagatgctgggggcagcagcgccacgtacatggaccaggccccctccccagctgtgtgcccccaggctccctataacatgtacccacagaaccctgaccatgtactcgatcaggatggagaattcgacctggatgagaccatggatgtggccaggcacgtggaggaactcttacgccgaccaatggacagtcttgactcccgcctctcgccccctgccggtcttttcacctctgccagaggctccctctcatgaatgtttgaatcccacgcttctctttggaaacaatatgcaatgtgaagcggtcgtgttgtgagtttagtaaggctgtgtacactgacacctttgcaggcatgcatgtgcttgtgtgtgtgtgtgtgtgtgtgtccttgtgcatgagctacgcctgcctcccctgtgcagtcctgggatgtggctgcagcagcggtggcctcttttcagatcatggcatccaagagtgcgccgagtctgtctctgtcatggtagagaccgagcctctgtcactgcaggcactcaatgcagccagacctattcctcctgggcccctcatctgctcagcagctatttgaatgagatgattcagaaggggaggggagacaggtaacgtctgtaagctgaagtttcactccggagtgagaagctttgccctcctaagagagagagacagagagacagagagagagaaagagagagtgtgtgggtctatgtaaatgcatctgtcctcatgtgttgatgtaaccgattcatctctcagaagggaggctggggttcattttcgagtagtattttatactttagtgaacgtggactccagactctctgtgaaccctatgagagcgcgtctgggcccggccatgtccttagcacaggggggccgccggtttgagtgagggtttctgagctgctctgaattagtccttgcttggctgcttggccttgggcttcattcaagtctatgatgctgttgcccacgtttcccgggatatatattctctcccctccgttgggccccagccttctttgcttgcctctctgtttgtaaccttgtcgacaaagaggtagaaaagattgggtctaggatatggtgggtggacaggggccccgggacttggagggttggtcctcttgcctcctggaaaaaacaaaaacaaaaaactgcagtgaaagacaagctgcaaatcagccatgtgctgcgtgcctgtggaatctggagtgaggggtaaaagctgatctggtttgactccgctggaggtggggcctggagcaggccttgcgctgttgcgtaactggctgtgttctggtgaggccttgctcccaaccccacacgctcctccctctgaggctgtaggactcgcagtcaggggcagctgaccatggaagattgagagcccaaggtttaaacttctctgaagggaggtggggatgagaagaggggtttttttgtactttgtacaaagaccacacatttgtgtaaacagtgttttggaataaaatatttttttcataaaaaaaaaaaaaaaa

SEQ ID NO: 12 - protein, 794 aa, Homo sapiens
MAGWIQAQQLQGDALRQMQVLYGQHFPIEVRHYLAQWIESQPWDAIDLDNPQDRAQATQLLEGLVQELQKKAEHQVGEDGFLLKIKLGHYATQLQKTYDRCPLELVRCIRHILYNEQRLVREANNCSSPAGILVDAMSQKHLQINQTFEELRLVTQDTENELKKLQQTQEYFIIQYQESLRIQAQFAQLAQLSPQERLSRETALQQKQVSLEAWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNECSGEILNNCCVMEYHQATGTLSAHFRNMSLKRIKRADRRGAESVTEEKFTVLFESQFSVGSNELVFQVKTLSLPVVVIVHGSQDHNATATVLWDNAFAEPGRVPFAVPDKVLWPQLCEALNMKFKAEVQSNRGLTKENLVFLAQKLFNNSSSHLEDYSGLSVSWSQFNRENLPGWNYTFWQWFDGVMEVLKKHHKPHWNDGAILGFVNKQQAHDLLINKPDGTFLLRFSDSEIGGITIAWKFDSPERNLWNLKPFTTRDFSIRSLADRLGDLSYLIYVFPDRPKDEVFSKYYTPVLAKAVDGYVKPQIKQVVPEFVNASADAGGSSATYMDQAPSPAVCPQAPYNMYPQNPDHVLDQDGEFDLDETMDVARHVEELLRRPMDSLDSRLSPPAGLFTSARGSLS

SEQ ID NO: 13 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 14 - protein, 189 aa, Homo sapiens
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP